SEQUENCE LISTING (6 sequences)

SEQ ID NO: 1 - DNA, 1415 bp, Bacillus (HU13)
  misc_feature (12)..(12): n is a, c, g, or t
gggcagcactcngaatcacggcggacgggtgagtacacgtgggcaacctgcctgtaagactgggataactccgggaaaccggagctaataccggatactatgtcaaaccgcatggtttgacattcaaagacggtttcggctgtcacttacagatgggcccgcggcgcagtaactagttggtgaggtaatggctcaccaaggcgacgatgcgtagccgacctgagagggtgatcggccacactgggactgagacacggcccagactcctacgggaggcagcagtagggaatcttccgcaatggacgaaagtctgacggagcaacgccgcgtgagtgatgaaggttttcggatcgtaaaactctgttgtcagggaagaacaagtgccggagtaactgccggcaccttgacggtacctgaccagaaagccacggctaactacgtgccagcagccgcggtaatacgtaggtggcaagcgttgtccggaattattgggcgtaaagcgcgcgcaggcggtttcttaagtctgatgtgaaagcccccggctcaaccggggagggtcattggaaactgggaaacttgagtgcagaagacgagagtggaattccacgtgtaccggtgaaatgcgtagagatgtggaggaacaccagtggcgaaggcgactctctggtctgaactgacgctgaggcgcgaaagcgtggggagcgaacaggattagataccctggtagtccacgccgtaaacgatgagtgctaagtgttagagggtttccgccctttagtgctgcagctaacgcattaagcactccgcctggggagtacggtagcaagattgaaactcaaaggaattgacgggggcccgcacaagcggtggagcatgtggtttaattcgaagcaacgcgaagaaccttaccaggtcttgacatcctttgccacttttagagatagaaggttccccttcgggggacaaagtgacaggtggtgcatggttgtcgtcagctcgtgtcgtgagatgttgggttaagtccagcaacgagcgcaacccttgatcttagttgccagcattcagttgggcactctaaggtgactgccggtgacaaaccggaggaaggtggggatgacgtcaaatcatcatgccccttatgacctgggctacacacgtgctacaatggatggtacaaagggctgcgagaccgcgaggtttagccaatcccataaaaccattctcagttcggattgcaggctgcaactcgcctgcatgaagctggaatcgctagtaatcgcggatcagcatgccgcggtgaatacgttcccgggccttgtacacaccgcccgtcacaccacgagagtttgcaacacccgaagtcggtggggtaaccgcaaggagccagccgcctaagggggtagtgatggatcctc

SEQ ID NO: 2 - DNA, 1407 bp, Bacillus (HU 16)
ccaggcctcgaatacgcggagggtgataccgtgggcacctgcctgtaagactgggataactccgggaaaccggagctaataccggatactatgtcaaaccgcatggtttgacattcaaagacggtttcggctgtcacttacagatgggcccgcggcgcagtaactagttggtgaggtaatggctcaccaaggcgacgatgcgtagccgacctgagagggtgatcggccacactgggactgagacacggcccagactcctacgggaggcagcagtagggaatcttccgcaatggacgaaagtctgacggagcaacgccgcgtgagtgatgaaggttttcggatcgtaaaactctgttgtcagggaagaacaagtgccggagtaactgccggcaccttgacggtacctgaccagaaagccacggctaactacgtgccagcagccgcggtaatacgtaggtggcaagcgttgtccggaattattgggcgtaaagcgcgcgcaggcggtttcttaagtctgatgtgaaagcccccggctcaaccggggagggtcattggaaactgggaaacttgagtgcagaagaggagagtggaattccacgtgtagcggtgaaatgcgtagagatgtggaggaacaccagtggcgaaggcgactctctggtctgtaactgacgctgaggcgcgaaagcgtggggagcgaacaggattagataccctggtagtccacgctgtaaacgatgagtgctaagtgttagagggtttccgccctttagtgctgcagctaacgcattaagcactccgcctggggagtacggtagcaagactgaaactcaaaggaattgacgggggcccgcacaagcggtggagcatgtggtttaattcgaagcaacgcgaagaaccttaccaggtcttgacatcctttgccacttctagagatagaaggttccccttcgggggacaaagtgacaggtggtgcatggttgtcgtcagctcgtgtcgtgagatgttgggttaagtcccgcaacgagcgcaacccttgatcttagttgccagcattcagttgggcactctaaggtgactgccggtgacaaaccggaggaaggtggggatgacgtcaaatcatcatgccccttatgacctgggctacacacgtgctacaatggatggtacaaagggctgcgagaccgcgaggtttagccaatcccataaaaccattctcagttcggattgcaggctgcaactcgcctgcatgaagctggaatcgctagtaatcgcggatcagcatgccgcggtgaatacgttcccgggccttgtacacaccgcccgtcacaccacgagagtttgcaacacccgaagtcggtggcgtaaccgcaaggagccagccgcctaaggggggtgtgatggatcctc

SEQ ID NO: 3 - DNA, 1420 bp, Bacillus (HU 19)
  misc_feature (12)..(12): n is a, c, g, or t
gggaggacctcngagtagcggcggacgggtgagtacacgtgggcaacctgcctgtaagactgggataactccgggaaaccggagctaataccggatactatgtcaaaccgcatggtttgacattcaaagacggtttcggctgtcacttacagatgggcccgcggcgcagtaactagttggtgaggtaatggctcaccaaggcgacgatgcgtatccgacctgagagggtgatcggccacactgggactgagacacggcccaagactcctaacggaaggcagcagtagggaatcttccgcaatggacgaaagtctgacggagcaacgccgcgtgagtgatgaaggttttcggatcgtagaactctgttgtcagggaagaacaagtgccggagtaactgccggcaccttgacggtacctgaccagaaagccacggctaactacgtgccagcagccgcggtaatacgtaggtggcaagcgttgtccggaattattgggcgtaaagcgcgcgcaggcggtttcttaagtctgatgtgaaagcccccggctcaaccggggagggtcattggaaactgggaaacttgagtgcagaagaggagagtggaattccacgtgtagcggtgaaatgcgtagagatgtggaggaacaccagtggcgaaggcgactctctggtctgtaactgacgctgaggcgcgaaagcgtggggagcgaacaggattagataccctggtagtccacgctgtaaacgatgagtgctaagtgttagagggtttccgccctttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctttgccacttctagagatagaaggttccccttcgggggacaaagtgacaggtggtgcatggttgtcgtcagctcgtgtcgtgagatgttgggttaagtcccgcaacgagcgcaacccttgatcttagttgccagcattcagttgggcactctaaggtgactgccggtgacaaaccggaggaaggtggggatgacgtcaaatcatcatgccccttatgacctgggctacacacgtgctacaatggatggtacaaagggctgcgagaccgcgaggtttagccaatcccataaaaccattctcagttcggattgcaggctgcaactcgcctgcatgaagctggaatcgctagtaatcgcggatcagcatgccgcggtgaatacgttcccgggccttgtacacaccgcccgtcacaccacgagagtttgcaacacccgaagtcggtggggtaaccgcaaggagccagccgcctaagggggtagatatggatgcttcc

SEQ ID NO: 4 - DNA, 1417 bp, Bacillus (HU 28)
  misc_feature (18)..(18): n is a, c, g, or t
gagcggactcttcggagntcagcggcggacgggtgagtaacacgtgggcaacctgcctgtaagactgggataactccgggaaaccggagctaataccggatactatgtcaaaccgcatggtttgacattcaaagacggtttcggctgtcacttacagatgggcccgcggcgcattagctagttggtgaggtaatggctcaccaaggcgacgatgcgtagccgacctgagagggtgatcggccacactgggactgagacacggcccagactcctacgggaggcagcagtagggaatcttccgcaatggacgaaagtctgacggagcaacgccgcgtgagtgatgaaggttttcggatcgtaaaactctgttgtcagggaagaacaagtgccggagtaactgccggcgccttgacggtacctgaccagaaagccacggctaactacgtgccagcagccgcggtaatacgtaggtggcaagcgttgtccggaattattgggcgtaaagcgcgcgcaggcggtttcttaagtctgatgtgaaagcccccggctcaaccggggagggtcattggaaactgggaaacttgagtgcagaagaggagagtggaattccacgtgtagcggtgaaatgcgtagagatgtggaggaacaccagtggcgaaggcgactctctggtctgtaactgacgctgaggcgcgaaagcgtggggagcgaacaggattagataccctggtagtccacgccgtaaacgatgagtgctaagtgttagagggtttccgccctttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctttgccacttctagagatagaaggttccccttcgggggacaaagtgacaggtggtgcatggttgtcgtcagctcgtgtcgtgagatgttgggttaagtcccgcaacgagcgcaacccttgatcttagttgccagcattcagttgggcactctaaggtgactgccggtgacaaaccggaggaaggtggggatgacgtcaaatcatcatgccccttatgacctgggctacacacgtgctacaatggatggtacaaagggctgcgagaccgcgaggtttagccaatcccataaaaccattctcagttcggattgcaggctgcaactcgcctgcatgaagctggaatcgctagtaatcgcggatcagcatgccgcggtgaatacgttcccgggccttgtacacaccgcccgtcacaccacgagagtttgcaacacccgaagtcggtggggtaaccgcaaggagccagccgcctaaggtggggtagagatgg

SEQ ID NO: 5 - DNA, 1416 bp, Bacillus (HU 33)
  misc_feature (1405)..(1405): n is a, c, g, or t
gcgaggcactcgaatacggcggagggtgagtacacgtgggcaacctgcctgtaagactgggataactccgggaaaccggagctaataccggatactatgtcaaaccgcatggtttgacattcaaagacggtttcggctgtcacttacagatgggcccgcggcgcattagctagttggtgaggtaatggctcaccaaggcgacgatgcgtagccgacctgagagggtgatcggccacactgggactgagacacggcccagactcctacgggaggcagcagtagggaatcttccgcaatggacgaaagtctgacggagcaacgccgcgtgagtgatgaaggttttcggatcgtaaaactctgttgtcagggaagaacaagtgccggagtaactgccggcgccttgacggtacctgaccagaaagccacggctaactacgtgccagcagccgcggtaatacgtaggtggcaagcgttgtccggaattattgggcgtaaagcgcgcgcaggcggtttcttaagtctgatgtgaaagcccccggctcaaccggggagggtcattggaaactgggaaacttgagtgcagaagaggagagtggaattccacgtgtagcggtgaaatgcgtagagatgtggaggaacaccagtggcgaaggcgactctctggtctgtaactgacgctgaggcgcgaaagcgtggggagcgaacaggattagataccctggtagtccacgccgtaaacgatgagtgctaagtgttagagggtttccgccctttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctttgccacttctagagatagaaggttccccttcgggggacaaagtgacaggtggtgcatggttgtcgtcagctcgtgtcgtgagatgttgggttaagtcccgcaacgagcgcaacccttgatcttagttgccagcattcagttgggcactctaaggtgactgccggtgacaaaccggaggaaggtggggatgacgtcaaatcatcatgccccttatgacctgggctacacacgtgctacaatggatggtacaaagggctgcgagaccgcgaggtttagccaatcccataaaaccattctcagttcggattgcaggctgcaactcgcctgcatgaagctggaatcgctagtaatcgcggatcagcatgccgcggtgaatacgttcccgggccttgtacacaccgcccgtcacaccacgagagtttgcaacacccgaagtcggtggggaaccgcaaggagccagccgcctaaggtgggtagatgntggaactgtcc

SEQ ID NO: 6 - DNA, 1416 bp, Bacillus (HU 36)
gggaggcacttggaatagcggcggacgggtgagtaaccgtgggcaacctgcctgtaagactgggataactccgggaaaccggagctaataccggatactatgtcaaaccgcatggtttgacattcaaagacggtttcggctgtcacttacagatgggcccgcggcgcattaactagttggtgaggtaatggctcaccaaggcgacgatgcgtagccgacctgagagggtgatcggccacactgggactgagacacggcccagactcctacgggaggcagcagtagggaatcttccgcaatggacgaaagtctgacggagcaacgccgcgtgagtgatgaaggttttcggatcgtaaaactctgttgtcagggaagaacaagtgccggagtaactgccggcgccttgacggtacctgaccagaaagccacggctaactacgtgccagcagccgcggtaatacgtaggtggcaagcgttgtccggaattattgggcgtaaagcgcgcgcaggcggtttcttaagtctgatgtgaaagcccccggctcaaccggggagggtcattggaaactgggaaacttgagtgcagaagacgagagtggaattccacgtgtagcggtgaaatgcgtagagatgtggaggaacaccagtggcgaaggcgactctctggtctgtaactgacgctgaggcgcgaaagcgtggggagcgaacaggattagataccctggtagtccacgccgtaaacgatgagtgctaagtgttagagggtttccgccctttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctttgccacttctagagatagaaggttccccttcgggggacaaagtgacaggtggtgcatggttgtcgtcagctcgtgtcgtgagatgttgggttaagtcccgcaacgagcgcaacccttgatcttagttgccagcattcagttgggcactctaaggtgactgccggtgacaaaccggaggaaggtggggatgacgtcaaatcatcatgccccttatgacctgggctacacacgtgctacaatggatggtacaaagggctgcgagaccgcgaggtttagccaatcccataaaaccattctcagttcggattgcaggctgcaactcgcctgcatgaagctggaatcgctagtaatcgcggatcagcatgccgcggtgaatacgttcccgggccttgtacacaccgcccgtcacaccacgagagtttgcaacacccgaagtcggtggggaaccgcaaggagccagccgcctaaggtggtagtgtggatgcttcc

(19) United States
(12) Patent Application Publication
(10) Pub. No.: US 2009/0175911 A1
(43) Pub. Date: Jul. 9, 2009

(54) Bacterial Production of Carotenoids

(75) Inventors: Simon Cutting, Surrey (GB); Paul Fraser, Surrey (GB)

Correspondence Address: NIXON & VANDERHYE, PC, 901 NORTH GLEBE ROAD, 11TH FLOOR, ARLINGTON, VA, 22203, US

(73) Assignee: ROYAL HOLLOWAY AND BEDFORD NEW COLLEGE, Surrey (GB)

(21) Appl. No.: 12/086,011

(22) PCT Filed: Dec. 6, 2006
(86) PCT No.: PCT/GB2006/004562

§ 371 Date: Sep. 23, 2008

(30) Foreign Application Priority Data
Dec. 6, 2005 (GB) 0524873.7

(51) Int. Cl. C12N 1/20; A61K 8/18; C12P 23/00; A61K 39/07; A61K 35/74; C12Q 1/22; A23K 1/16; A23K 1/18

(52) U.S. Cl. 424/246.1; 424/93.46; 8/636; 424/93.2; 435/67; 435/252.5; 424/780; 435/5; 426/2; 426/61

(57) ABSTRACT

The present invention is based on a Bacillus the spores and vegetative cells forms of which are a different colour because of differential presence of at least one carotenoid in the spore and vegetative cell forms of the Bacillus. The Bacillus may therefore be used in detection methods and biosensors. The Bacillus may also be used a colourant and a dye and in the generation of foods, food supplements, probiotic compositions, dyes, cosmetic, pharmaceuticals and vaccines. The Bacillus may also be used for the production of carotenoids, precursors thereof and downstream derivatives.

### FIELD OF THE INVENTION

The present invention relates to non-pathogenic spore-forming Bacilli. In particular, the invention relates to the use of the Bacilli in, or as, foods, food supplements, probiotics, colourants, dyes, biosensors, sources of carotenoid and isoprenoid derived metabolites, as well as to the Bacilli themselves. The invention also relates to using the Bacilli in methods of detecting stimuli.

### BACKGROUND OF THE INVENTION

Carotenoids are the most widespread group of naturally occurring pigments found in nature; these yellow, orange and red coloured molecules are found in both eukaryotes and prokaryotes. At least 600 structurally different compounds are now known, with an estimated yield of 100 million tonnes per annum (Britton et al., 2003). One of the principal functions of carotenoids within the cell is to provide protection against photo-oxidative damage by quenching singlet oxygen as well as other harmful radicals that are formed when cells are illuminated. In photosynthetic organisms they play a vital role as light-harvesting pigments, while in mammals the cleavage of some carotenoids (e.g., β-carotene) plays an important role in nutrition (Vitamin A), vision (retinal) and development (retinoic acid). In addition, it is the inherent potent antioxidant properties of carotenoids that protect cells from environmental extremes and in mammals can prevent the onset of chronic disease states. These health-promoting properties have lead to substantial interest in carotenoids as nutritional supplements, particularly as mammals (most notably humans) cannot synthesise carotenoids de novo and they must be acquired from the diet.

Commercially, carotenoids are used in the pharmaceutical, cosmetic, and food and feed industries, particularly as precursors, colorants and supplements. The global market is expanding and in 2005 has been estimated at $935 million. Total chemical synthesis is the method of choice at present for producing carotenoids industrially. The disadvantages of this approach include the production of stereo isomers not found in the natural product, contamination with reaction intermediates/products and lack of potential synergistic nutrients present in biological mixtures. Thus there is a need for carotenoid production from natural sources.

Microbial sources of carotenoids presently used commercially include the unicellular algae Dunaliella salina, Spirulina and Haematococcus, the yeast Phaffia rhdozyma and filamentous fungi Blakeslea trispora and Phycomyces blakesanus. However the unicellular algae are slow growing, prone to contamination, require high oxygenation rates, and intense light. These conditions have limited production sites to areas of Hawaii and Australia. Both the Phaffia rhdozyma and Phycomyces blakesanus are comparatively slow growing in comparison to bacteria and require cooled fermentation conditions which has important cost implications. Blakeslea trispora also has a slower growth rate compared to potential bacterial sources and require sexual stimulation by trisporic acids for high yields. At present there is only one higher plant source (Tagetes flowers) from which carotenoids are produced commercially.

### SUMMARY OF THE INVENTION

The present invention provides for the use of a Bacillus, or an extract from a Bacillus, as a colourant, dye, food additive, food supplement or in a cosmetic, wherein the Bacillus has, or the extract is obtained from, a Bacillus with, at least 95% 16S rRNA sequence identity to the sequence of any of SEQ ID Nos 1 to 6.

The invention also provides a method of producing a carotenoid, a metabolic precursor thereof or a derivative thereof, comprising: (i) growing vegetatively and/or producing spores of a Bacillus of the invention; and (ii) extracting the carotenoid, metabolic precursor thereof, or the derivative thereof from the Bacillus.

The present invention provides a method of detecting a stimulus comprising:
(i) providing spores of a Bacillus which are triggered when the stimulus is present to germinate to give vegetative cells, where the spores and the vegetative cells of the Bacillus are a different colour because of differential presence of at least one carotenoid in the spore and vegetative cell forms of the Bacillus;
(ii) exposing the spores to test conditions under which it is desired to determine whether the stimulus is present or absent; and
(iii) detecting the presence or absence of the colour change resulting from germination of spores in order to determine the presence or absence of the stimulus.

The invention also provides for the use of Bacillus, or an extract from a Bacillus, as a colourant, dye, food additive, food supplement or in a cosmetic where the Bacillus, or the extract is obtained from a Bacillus, where the spores and vegetative cells of the Bacillus are a different colour because of the differential presence of at least one carotenoid.

The invention also provides a method of detecting an agent capable of modulating Bacillus growth, germination or sporulation, the method comprising contacting a test agent with a Bacillus of the invention and monitoring for a colour change or change in colour intensity, where the spores and the vegetative cells of the Bacillus are a different colour because of differential presence of at least one carotenoid in the spore and vegetative cell forms of the Bacillus.

The invention provides a Bacillus selected from a Bacillus with at least 95% 16S rRNA sequence identity to the sequence of any one of SEQ ID Nos 1 to 6, where the spores and vegetative cells of the Bacillus are a different colour because of the differential presence of at least one carotenoid in the spore and vegetative cell forms

The invention additionally provides a Bacillus selected from Bacillus spp. HU19 (NCIMB 41359), HU28 (NCIMB 41360), HU33 (NCIMB 41342) and HU36 (NCIMB 41361), or a derivative, variant or mutant of any thereof, where:
the spores and vegetative cells of the derivative, variant or mutant Bacillus are a different colour because of differential presence of at least one carotenoid in the spore and vegetative cells forms; and
the 16S rRNA gene of the derivative and mutant has at least 95% 16S rRNA sequence identity to that of any one of HU 19, HU 28, HU 33, and HU 36.

Also provided is:
a biosensor comprising a Bacillus of the invention and a support, where the spores and the vegetative cells of the Bacillus are a different colour because of differential presence of at least one carotenoid in the spore and vegetative cell forms of the Bacillus;
a food-stuff, food additive, dye, colourant, cosmetic, nutraceutical or probiotic composition comprising a Bacillus of the invention, or an extract from a Bacillus of the invention;
a pharmaceutical comprising a Bacillus of the invention, or an extract from such a Bacillus, and a pharmaceutically acceptable carrier or excipient; and
a Bacillus of the invention or a carotenoid extract from such a Bacillus for use in a method of treatment of the human or animal body.

### BRIEF DESCRIPTION OF THE FIGURES

FIG. 1 shows the neighbour-joining trees of the Bacillus clones HU 19, HU 28, HU 36 and HU 33, based on 16S rRNA (rrnE) analysis. Values of sequence identity are shown in brackets. Also shown are other related species with strain designations. The strains above the dotted line all have at least 99% rRNA sequence identity to each other.

FIGS. 2A-2F show spectra of various extracts. UV/Vis spectra recorded at 450 nm: 3A, PY79 wild type; 3B, Spores of HU 36; 3C, vegetative material of HU 36. UV spectra recorded at 286 nm; 3D, PY79 wild type; 3E, Spores of HU 36; and 3F, vegetative material of HU 36; each peak numbered in 3B and 3C indicates characteristic carotenoid signature spectrum. Peak 13—represents phytoene and 14-ubiquinone.

FIG. 3 shows the quantification of carotenoids, UBQ-ubiquinone, HDMS-hydroxy-demethylspheroidene, KHGC-keto/hydroxy β-carotene, DMS-demethylspheroidene and OS-3,4-dihydrospheroidene which are found in the Bacillus clones HU 19 and HU 36 as well as the control strain PY79.

FIG. 4 shows the putative pathways involved in carotenoid formation during vegetative growth and spore formation. Those reactions that appear unique to carotenogenesis in spores are shown as dashed arrows.

FIG. 5 shows possible compounds that may be produced using the Bacilli of the invention.

FIG. 6 shows an example of a possible format for a biosensor of the invention. The biosensor (10) has a silicon substrate (20) on which are placed 500×10⁶ CFU of a spore (30) of the bacterium according to the invention prepared as described in Example 6.

FIG. 7 shows the percentage carotenoid remaining after incubation with Simulated Gastric Fluid (SGF) for 0, 20 and 60 minutes with the columns representing, from left to right, HU36 spores (c), a carotenoid extract from HU36 spores (EC) and a beta carotene standard (CS).

FIG. 8 Shows the adhesion of Bacillus strains to Caco-2, HEp-2 and HT29 cell lines. Spores (1×10⁸) of the indicated strains were incubated with cell lines at 37° C. in the presence of Gentamycin (100 mg/ml). The percentage of spores adhering to the different cell lines was determined after two hours incubation.

### BRIEF DESCRIPTION OF THE SEQUENCES

SEQ ID Nos 1 to 6 provide the nucleotide sequences of the 16S rRNA genes of the HU 13, 16, 19, 28, 33 and 36 isolates.

### DETAILED DESCRIPTION OF THE INVENTION

The present invention provides various Bacilli and for their uses. In particular, the Bacilli produce carotenoids making them useful for the production of carotenoids. The Bacilli, and extracts therefrom, can be used, for instance, as colourants, dyes, food additives, food supplements and in cosmetics.

The present invention typically involves Bacilli that comprise at least one carotenoid that is present in a different amount in their spore and vegetative cell forms. In particular, the invention may involve the use of Bacilli whose:

(i) spores comprise at least one carotenoid which is not present in the vegetative cell form of the Bacillus; and/or

(ii) vegetative cells comprise at least one carotenoid which is not present in the spore form of the Bacillus.

In one particularly important embodiment, the Bacilli, or extracts thereof, may be used in aquaculture, particularly being given to crustaceans and especially shellfish. Such uses can result in increased yields.

The Bacilli may be put to a wide variety of uses. As carotenoids are coloured, in one instance the spore and vegetative cell forms of the Bacilli may be different colours and the colour difference of the two forms can be used in methods to detect various stimuli and also in biosensors to detect such stimuli. The colour of the carotenoids means that the Bacilli, and extracts from them comprising the carotenoids, can be used as colourants and dyes. They may be also be used in foods and food additives, both to add colour to them and also for their probiotic and anti-oxidant functions. In some embodiments, the Bacillus may not display a colour change between spore and vegetative states.

The Bacilli, and extracts from them, may be fed to farmed animals, both land animals and marine animals, in particular to provide colour in one or more tissues of the organism. In another instance, they may be additionally, or alternatively, fed to increase yields. The Bacilli, and materials obtained from them, may also be used in cosmetics, pharmaceutical compositions, as probiotics and as competitive exclusion agents.

Carotenoids and various metabolic precursors and derivatives thereof are also commercially valuable materials and hence the Bacilli may be used as cell factories to produce such materials and the desired compounds then extracted from the Bacillus.

### Bacilli

The present invention is based on the use of Bacilli that typically have at least one carotenoid present in a different amount in the vegetative and spore forms of the Bacillus. The Bacillus is typically a different colour in its spore and vegetative forms due to the differential presence of at least one carotenoid in the two forms. That is, a particular carotenoid may be present in the spore form in a higher amount than in the vegetative form of the Bacillus and/or a carotenoid may be present in the vegetative form of the Bacillus in a higher amount than in the spore form. In an alternative embodiment, the Bacillus may not display such a colour change between spore and vegetative states.

In one instance, the Bacillus will have at least 95% 16S rRNA sequence identity to any of SEQ ID Nos 1 to 6. In one preferred embodiment, the Bacillus employed may be one selected from Bacillus spp. HU19 (NCIMB 41359), HU28 (NCIMB 41360), HU33 (NCIMB 41342) and HU36 (NCIMB 41361), or a derivative, variant or mutant of any thereof, where the 16S rRNA gene of the derivative, variant or mutant has at least 95% 16S rRNA sequence identity to that of any one of HU 19, HU 28, HU 33, and HU 36.

In some instances, one or more carotenoids may be present in higher amounts in the spore form than the vegetative form and there may also be one or more different carotenoids that are present in higher amounts in the vegetative form in comparison to the spore form of the Bacillus.

The difference in the amount of carotenoid may, for instance, be double, preferably at least triple, preferably at least 5 times, more preferably at least 10 times, more preferably at least 25 times, more preferably at least 50 times, even more preferably at least 100 times and still more preferably at least 1000 times the amount it is present in the other form. In some embodiments, such multiples may represent the upper limit of the increase.

In a particularly preferred instance, there is substantially none of a particular carotenoid present in one form of the Bacillus which is present in the other form of the Bacillus and preferably one or more carotenoids are present in the spore or vegetative form, but are completely absent from the other form.

Any suitable technique may be used to measure carotenoid levels and hence compare levels in the spores and vegetative cells of a Bacillus. In a preferred technique, equal weights of lyophilised spores and vegetative cells are ground, such as in a pestle-and-mortar, and then chloroform extracted. Typically the ground material is dissolved in one volume of methanol and two of chloroform, incubated on ice for 10 to 30 minutes, preferably for about 20 minutes, a volume of water equivalent to the volume of methanol added, the sample vortexed and, after allowing to settle, the organic fraction recovered by removal of the aqueous phase. The sample, may then typically be twice more extracted and then the organic phase reduced to complete dryness under a stream of nitrogen. The relative amounts of carotenoids may then be analysed, for instance spectrophotometry may be employed, preferably combined with HPLC separation.

In one instance, the amount of carotenoids may be measured using HPLC and measuring at 450 nm and/or at 286 nm and in particular at 450 nm to compare levels. The techniques described herein, may, for instance, be employed for the isolation of carotenoids and HPLC analysis. The analysis in typically done by comparing the results for an extract from an equal weight of lyophilised spores and vegetative cells. In one instance, the colour of the spores and vegetative cells may be compared as a guide to differential carotenoid presence, for instance, by the naked eye. Sporulation may be induced and a colour change monitored for. Colony colour may be examined.

The Bacillus may produce one, preferably at least two, more preferably at least four, more preferably at least six, more preferably at least eight, still more preferably at least ten and still more preferably eleven or more carotenoids which are present in higher amounts in the spore form than in the vegetative cells or vice versa.

In preferred instances, the Bacillus employed may be one selected from Bacillus subtilis, Bacillus amyloliquifaciens, Bacillus pumilus, Bacillus licheniformis, Bacillus circulans, Bacillus coagulans, Bacillus firmus, Bacillus jeotgali, Bacillus clausii, Bacillus pseudofirmus, Bacillus okuhidensis, Bacillus clarkia, Bacillus vedderi, Bacillus megaterium, Bacillus flexus, Bacillus cohnii, Bacillus indicus, Bacillus cibi and Bacillus catenulatus. That may particularly be the case where the Bacillus is one whose spores and vegetative cells are a different colour.

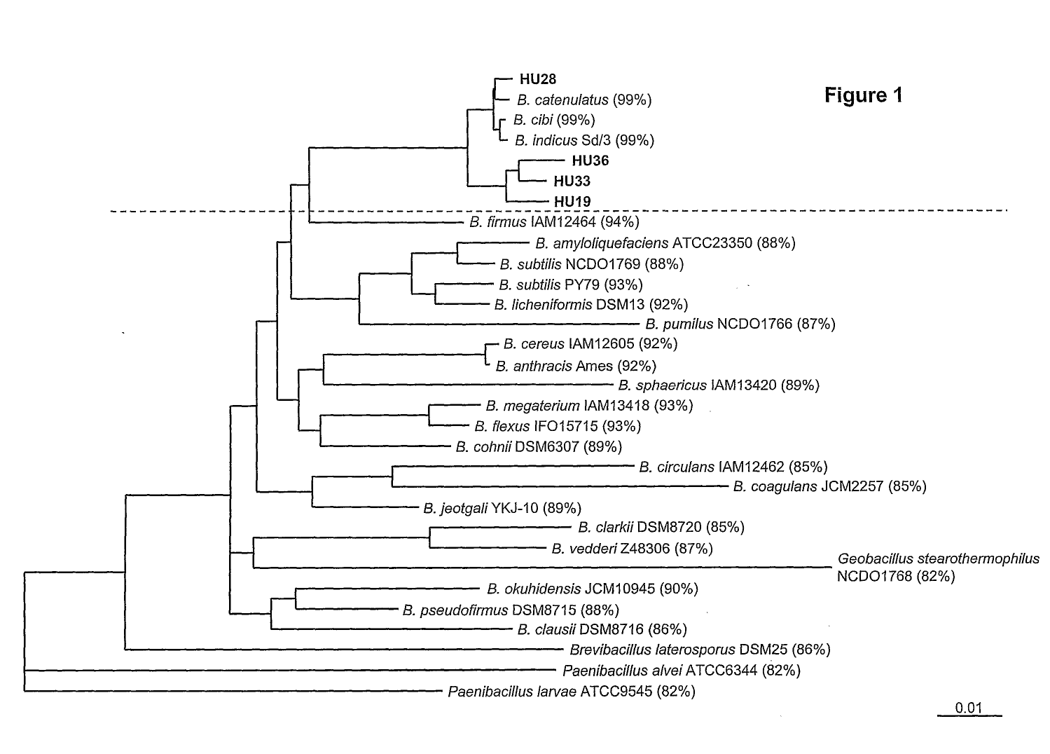

In one embodiment the Bacillus may be any of those indicated in FIG. 1, particularly one which displays a colour change between spore and vegetative forms. In one preferred instance, the Bacillus may be one selected from Bacillus cibi, Bacillus indicus, Bacillus catenulatus and the deposited strains discussed herein and variants or mutants thereof which have at least 95% sequence identity at the 16S rRNA level. In an alternative instance, the Bacillus provided may exclude Bacillus cibi, Bacillus indicus and Bacillus catenulatus. In one particularly preferred instance, the Bacillus will display the colour change between spore and vegetative forms. In others it may not.

In a particularly preferred instance, the Bacillus employed may be one of a closely related group of Bacilli that can be defined by reference to 16S rRNA sequence identity. For instance, the Bacillus may be one that displays at least 95% sequence identity to any one of the 16S rRNA sequences of SEQ ID Nos 1 to 6.

In one preferred instance, the Bacillus may be one that: (i) has at least one carotenoid present in a different amount in the vegetative or spore form of the Bacillus in comparison to the other form of the Bacillus; and (ii) whose 16S rRNA has at least 90% sequence identity to any one of the polynucleotide sequences of SEQ ID NOS: 1 to 6.

In a preferred instance, the 16S rRNA of the Bacillus may have at least 91%, preferably at least 92%, more preferably at least 93%, and even more preferably at least 94% sequence identity to the polynucleotide sequences of any one of SEQ ID NOS: 1 to 6. In a particularly preferred instance, the 16S rRNA sequence of the Bacillus may have at least 95%, preferably at least 96%, more preferably at least 97%, more preferably at least 98% and even more preferably at least 99% sequence identity to any one of SEQ ID NOS: 1 to 6. In particular, the 16S rRNA of the Bacillus may have at least 99.25%, preferably at least 99.5%, more preferably at least 99.75% and still more preferably 99.9% sequence identity to any one of SEQ ID NOS: 1 to 6. In a particularly preferred instance the level of sequence identity will be to any one of SEQ ID NOS: 3 to 6. In one preferred instance, the level of sequence identity may be to any one of SEQ ID NOS: 3, 4 and 6 and in an alternative instance to SEQ ID NO: 5.

A variety of programs may be used to calculate percentage homology. The UWGCG Package provides the BESTFIT program which can be used to calculate homology (for example used on its default settings) (Devereux et al (1984) Nucleic Acids Research 12, p 387-395). The PILEUP and BLAST algorithms can be used to calculate homology or line up sequences (typically on their default settings), for example as described in Altschul S. F. (1993) J Mol Evol 36:290-300; Altschul, S. F et al (1990) J Mol Biol 215:403-10.

Software for performing BLAST analyses is publicly available through the National Centre for Biotechnology Information (http://www.ncbi.nlm.nih.gov/). This algorithm involves first identifying high scoring sequence pair (HSPs) by identifying short words of length W in the query sequence that either match or satisfy some positive-valued threshold score T when aligned with a word of the same length in a database sequence. T is referred to as the neighbourhood word score threshold (Altschul et al, supra); These initial neighbourhood word hits act as seeds for initiating searches to find HSPs containing them. The word hits are extended in both directions along each sequence for as far as the cumulative alignment score can be increased. Extensions for the word hits in each direction are halted when the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLAST program uses as defaults a word length (W) of 11, the BLOSUM62 scoring matrix (see Henikoff and Henikoff (1992) Proc. Natl. Acad. Sci. USA 89: 10915-10919) alignments (B) of 50, expectation (E) of 10, M=5, N=4, and a comparison of both strands.

The BLAST algorithm performs a statistical analysis of the similarity between two sequences; see e.g., Karlin and Altschul (1993) Proc. Natl. Acad. Sci. USA 90: 5873-5787. One measure of similarity provided by the BLAST algorithm is the smallest sum probability (P(N)), which provides an indication of the probability by which a match between two nucleotide or amino acid sequences would occur by chance. For example, a sequence is considered similar to another sequence if the smallest sum probability in comparison of the first sequence to the second sequence is less than about 1, preferably less than about 0.1, more preferably less than about 0.01, and most preferably less than about 0.001.

In one instance, levels of sequence identity may be over at least 10%, preferably at least 25%, more preferably at least 50%, even more preferably at least 75% and still more preferably over at least 95% of the length of the 16S rRNA gene. In a particularly preferred instance, sequence identity may be measured over the entire length, or almost the entire length, of the 16S rRNA gene. Sequence identity may, in some cases, be measured over at least 50, preferably at least 100, and even more preferably over at least 250 nucleotides, more preferably at least 500 nucleotides, still more preferably at least 1000 nucleotides and even more preferably at least 1400 nucleotides.

In one preferred instance, phylogenetic analysis may be used to define a preferred Bacillus strain for use in the invention and in particular such analysis of the 16S rRNA gene may be used. Phylogenetic trees may be drawn up using the CLUSALW programme (available at http://align.genome.jp). The methodology employed by Ash et al (1991) may be employed in the phylogenetic analysis. In one preferred instance, the Bacillus is a Group 1 Bacillus as defined by the phylogenetic analysis of the 16S rRNA of the Bacillus as defined by the methodology described herein. In another particularly preferred embodiment the Bacillus is closer to the 16S rRNA sequence of any one of SEQ ID NOS: 1 to 6, Bacillus catenulatus, Bacillus cibi and Bacillus indicus in phylogenetic analysis than to Bacillus circulans, preferably than to Bacillus sphaericus and in particular than to Bacillus firmus. In one instance, the Bacillus would fall within the same phylogentic grouping as Bacillus catenulatus, Bacillus cibi and Bacillus indicus and the sequences of SEQ ID NOS: 1 to 6.

In one preferred instance the phylogentic analysis is performed with CLUSTALW and in particular version 1.83 (http://align.genome.jp/). Bar is preferably set at 0.01 nucleotide substitutions per site. Percentage similarity may preferably be determined by CLUSTALW multiple alignment [http://npsa-pbil.ibcp.fr/]programme) of the sequences.

In one preferred embodiment of the invention the bacterium according to the invention is preferably Bacillus spp. HU33 (NCIMB 41342). NCIMB 41342 was deposited on 19 Sep. 2005. In a particularly preferred instance the bacterium according to the invention is preferably one of Bacillus spp. HU19 (NCIMB 41359), HU28 (NCIMB 41360) and HU36 (NCIMB 41361). NCIMB 41359, 41360 and 41361 were deposited on 1 Dec. 2005. In a particularly preferred instance, the Bacillus may be HU36 or a variant, derivative or mutant thereof. NCIMB—National Collections of Industrial, Marine and Food Bacteria, Ferguson Building, Craibstone Estate, Bucksburn, Aberdeen, AB219YA, UK.

In one instance, the Bacillus is:
(i) one of Bacillus spp. HU19, HU28, HU33 or HU36 having accession numbers NCIMB 41342, 41359, 41360 and 41361 respectively; and/or
(ii) a variant strain or mutant strain thereof which has at least 90% sequence identity to the 16S rRNA sequence of any one of the said four strains.

In a preferred instance the level of sequence identity is at least 95%.

The bacterium according to the invention is typically coloured. The colour of the bacterium according to the invention in its spore state is typically different from the colour of the bacterium according to the invention in its vegetative cell state. The colour of the bacterium according to the invention is preferably red, orange or yellow, especially orange or yellow. In particular, the bacterium is preferably yellow whilst growing vegetatively and orange in its spore state. Modifications may be made to the genes involved in carotenoid synthesis to produce different coloured Bacilli.

Typically, the Bacillus employed will display a colour switch between its vegetative and spore states, thus sporulation or germination will lead to a change in colour. In a particularly preferred instance, the colour change will be from orange to yellow or vice versa. In one instance, colour may be measured by the naked eye, in other instances colour change may, for instance, be measured by spectrophotometry or other similar methods. In some instances, the Bacillus may not display the colour change between the spore and vegetative states.

The Bacillus employed in the invention may also, or alternatively, show one or more of the following characteristics:
ellipsoidal spores within swollen sporangia;
be non-motile;
be able to hydrolyse starch;
be able to grow in the presence of sodium chloride, for instance, in at least 2% NaCl, preferably at least 4% NaCl, even more preferably at least 6% NaCl and still more preferably up to 8% NaCl solutions;
display tolerance to arsenate, for instance being able to grow in 2 mM arsenate, preferably 5 mM arsenate, more preferably at least 10 mM arsenate, still more preferably at least 15 mM arsenate and more preferably still in up to 20 mM arsenate;
be unable to grow anaerobically, particularly when streaked on nutrient rich agar; and/or
be unable to grow at a temperature of 50° C. or more.

In one instance, the Bacillus may display all of the above characteristics. In others, the Bacillus may display at least two, three, four, five or more of those characteristics. The Bacillus may in some instances be one which is able to grow anaerobically when grown in liquid culture, then streaked on DSM plates. The Bacillus may be one unable to sporulate under anaerobic conditions. In some embodiments the Bacillus may be one isolated, or originally isolated, from an aquatic organism or faecal matter from a subject and in particular from a human subject who has been consuming sea-food.

In a preferred instance, a Bacillus of the invention will comprise one or more of the following genes:

a ggpp synthase (crtE like) gene;

a phytoene synthase (crtB like) gene;

a three or four step phytoene desaturase (crtI like) gene;

a cyclase (probably mono-cyclase) (crtY like) gene;

a β-ring 3,3′ hydroxylase (crtZ like) gene; and/or

a β-ring 4,4′ oxygenase (ketolase), (crtW and crtO/bkt like) gene.

In some instances, the Bacillus may comprise all of the above genes, or at least two, three, four or five of the genes. Preferably, the Bacillus according to the invention has all of the above identified genes.

The Bacillus of the invention may be a naturally occurring variant of a deposited strain or may alternatively be a strain derived from a deposited strain or a mutant of such strains. Such natural variants and derived strains may retain any of the features mentioned herein. In a preferred instance, the 16S rRNA of the variant will have, or have at least, one of the percentage levels of sequence identity indicated herein to the Bacillus spp. HU 19, HU 28, HU 33 and HU 36 having accession numbers NCIMB 41359, 41360, 41361 and 41362 respectively. In another instance, the variant, mutant or derivative may have any of the levels of sequence identity referred to herein to any one of SEQ ID NOS: 1 to 6.

In one instance, the Bacillus of the invention may be grown with nitrate and/or nitrite. In one instance the Bacillus may lack any episome conferring antibiotic resistance. In one instance, the Bacillus may display the antibiotic resistance shown in Tables 9 and/or 10, particularly the profile, rather than the exact levels. The Bacillus may show resistance to clindamycin, it may not in an alternative embodiment. The strain may display low levels of adhesion to Caco-2 cells.

In one instance, any of the assays or techniques discussed in the Examples may be used to measure a particular parameter. The Bacillus may lack enterotoxins, particularly having no B. cereus enterotoxin genes. The Bacillus may persist for under two weeks in the GIT (gastrointestinal tract) as measured by analysing faeces.

The Bacillus is typically in the form of a spore or a vegetative cell, or a mixture of both. In a particularly preferred instance, the Bacillus is in the form of a spore. In instances where the Bacillus is in the form of a spore, it may have been treated such that it cannot germinate. For example, the spore could be treated by heat and may, for instance, have been subjected to autoclaving to prevent germination. In many instances, the Bacillus may be provided in the form of spores which can germinate. The spore or vegetative cell may, in one embodiment, be provided in isolated form.

### Modified Bacilli

In some instances, the Bacillus may be one that has been genetically modified and in particular one that has been genetically modified to increase its suitability for one of the uses described herein. Techniques for genetic modification are described in, for instance, Sambrook, J. and Russel D W. (Editors) Molecular Cloning, A laboratory Manual vol. 1, 2, and 3. Cold Spring Harbor Laboratory Press, Cold Spring Harbor, N.Y. 2001. third Edition.

The Bacillus may have been, for instance, subjected to random or directed mutagenesis and in particular selection may then have been performed to isolate mutants with desirable characteristics. The invention also provides a Bacillus which has been genetically modified so that it comprises a heterologous gene. In some instances, the Bacillus may have been modified by the introduction of one or more foreign nucleic acid sequence and in particular one or more genes. For instance, one, two, three, four, five or more foreign genes may have been introduced. Such genetic modifications may have been made so that the Bacillus produces a desired compound, or more of a desired compound. In some instances, a Bacillus may have been modified to mutate such numbers of genes, for instance to inactivate them or to mutate particular polypeptides.

The Bacillus may have been subjected to random mutagenesis. For instance, the bacterium may have been subjected to physical mutagenesis techniques such as X-ray radiation and UV radiation or they may be subjected to chemical mutagenesis agents such as N-methyl-N′-nitro-N-nitrosoguanidine (NTG), and ethylmethane sulfonate (EMS).

In some instances, the genetic modification may mean that the Bacillus produces a particular carotenoid that it did not produce previously or alternatively produces a particular carotenoid in higher or lower amounts than it had done previously and preferably in higher amounts. In other embodiments the modification may mean a desired precursor or derivative of a carotenoid or another metabolite is produced which had not been produced previously, or is produced in greater or lesser amounts, than had been produced previously, preferably in greater amounts. The Bacillus may therefore, in one instance, have a mutation in a gene involved in carotenoid synthesis, or have an additional heterologous gene involved in carotenoid synthesis introduced.

In one instance, the modification or variation may mean that the Bacillus does not display a colour change between spore and vegetative forms. In others, such a change in colour may be retained.

The introduced genes may be under the control of promoters and/or regulatory elements to give the desired expression. For instance, expression of genes may be made inducible so that a gene product is produced only when desired. Alternatively, other regulatory sequences may be present which target proteins. For instance, the foreign protein may be targeted to the spore coat and may, in a preferred instance, comprise a fusion protein with a coat protein of the spore or part of a coat protein in a spore. In a particularly preferred instance, the protein may be expressed as a fusion with the Cot B or CotC Bacillus coat proteins and in particular where the protein is an antigen. Foreign proteins expressed in the Bacilli may include enzymes, antigens, hormones and structural proteins.

In instances where a gene is introduced into the Bacillus that is not a Bacillus gene, the gene may be condon optimised for expression in the Bacillus.

In one instance, the Bacillus will comprise one or more of the following foreign genes:

a bicyclic beta cyclase (which is a product of the crtY and/or crtL-b genes);

an epilson cyclases (which is a product of the crtL-e gene);

a dehydrosqualene synthase (which is a product of the crtM gene);

a dehydrosqualene desaturase (which is a product of the crtN gene);

a phytoene desaturase (which is a product of the crtI gene):

a phytoene desaturase (which is a product of the Pds gene);

a ζ-carotene desaturase (which is a product of the crtQ gene);

a ζ-carotene desaturase (which is a product of the Zds gene);

a lycopene elongase (which is a product of the crtEb; C50 carotenoid biosynthesis);

a zeaxanthin glucosylase (which is a product of the crtX gene);

a C3 carotene hydroxylase (which is a product of crtZ like gene);

a C2 carotene hydroxylase (which is a product of the BcrtG gene);

a C4 oxygenase (which is a products of crtW like genes);

a C4 oxygenase (which are products of bkt like genes);

a zeaxanthin epoxidase (which is a product of the zep1 gene);

a violaxanthin deepoxidase (which is a product of the vde gene);

a capsanthin/capsorubin synthase (which is a product of the ccs gene);

a β-carotene desaturase (which is a product of the crtU gene);

a decaprenoxanthin synthase (which is a product of the crtYe/Yf gene); and/or

a carotenoid cleavage dioxygenase (CCD's) to yield aroma, flavour, and/or a pharmaceutical related compound (e.g retinol).

The close homology between various Bacilli facilitates the use of directed evolution, sexual PCR and gene shuffling (Schmidt-Dannert, C. 2000). Directed evolution of single proteins, metabolic pathways and viruses. Biochemistry 40, 13125-13136.) for the generation of improved enzyme activities and altered activities capable generating new carotenoid combinations.

Such modifications may be made to any of the Bacilli discussed herein, in a preferred instance to a Bacillus with at least 95% 16S rRNA sequence identity to any of SEQ ID Nos 1 to 6. Any of the modifications discussed herein may, in one instance, be made to the strains HU19, HU28, HU33, or HU36. The invention provides modified versions of those strains.

### Vaccines

The Bacilli may also be modified to express an antigen and hence be used as vaccines. Thus, the invention provides a vaccine comprising a Bacillus and a pharmaceutically acceptable carrier or excipient. In a particularly preferred instance the vaccines will be suitable for oral administration and in particular will be in spore form. In a preferred instance, the antigen is expressed on the surface of the vaccine. In another instance, it may be expressed intracellulary. The stability of spores means that they are particularly useful for the production of vaccines for developing countries where temperature control to preserve vaccines may not be readily possible or be too expensive. The colour of the spores also means that the vaccines are attractive for children.

Thus, the Bacillus may encode an antigen, immunogenic fragment thereof or immunogenic variant of either. The antigen may in particular be a viral, bacterial, parasitic or fungal pathogen antigen or a tumour antigen. In a preferred instance, the antigen may be a viral antigen, an immunogenic fragment thereof or an immunogenic variant of either.

The Bacillus may encode an antigen and hence be used as a vaccine for the treatment or prevention of a number of conditions including, but not limited to, cancer, allergies, toxicity and infection by a pathogen such as, but not limited to, fungus, viruses including Human Papilloma Viruses (HPV), HIV, HSV2/HSV1, influenza virus (types A, B and C), Polio virus, RSV virus, Rhinoviruses, Rotaviruses, Hepatitis A virus, Norwalk Virus Group, Enteroviruses, Astroviruses, Measles virus, Para Influenza virus, Mumps virus, Varicella-Zoster virus, Cytomegalovirus, Epstein-Barr virus, Adenoviruses, Rubella virus, Human T-cell Lymphoma type I virus (HTLV-I), Hepatitis B virus (HBV), Hepatitis C virus (HCV), Hepatitis D virus, Pox virus, Marburg and Ebola; bacteria including Mycobacterium tuberculosis, Chlamydia, N. gonorrhoeae, Shigella, Salmonella, Vibrio Cholera, Treponema pallidua, Pseudomonas, Bordetella pertussis, Brucella, Franciscella tulorensis, Helicobacter pylori, Leptospria interrogaus, Legionella pnumophila, Yersinia pestis, Streptococcus (types A and B), Pneumococcus, Meningococcus, Hemophilus influenza (type b), Toxoplama gondii, Complybacteriosis, Moraxella catarrhalis, Donovanosis, and Actinomycosis; fungal pathogens including Candidiasis and Aspergillosis; parasitic pathogens including Taenia, Flukes, Roundworms, Amebiasis, Giardiasis, Cryptosporidium, Schitosoma, Pneumocystis carinii, Trichomoniasis and Trichinosis. In a preferred instance, the vaccine may be one against Bacillus anthracis and in particular encode the Protective Antigen of Bacillus anthracis.

The Bacillus may also be used to provide a suitable immune response against numerous veterinary diseases, such as Foot and Mouth diseases, Coronavirus, Pasteurella multocida, Helicobacter, Strongylus vulgaris, Actinobacillus pleuropneumonia, Bovine viral diarrhea virus (BVDV), Klebsiella pneumoniae, E. coli, Bordetella pertussis, Bordetella parapertussis and Bordetella brochiseptica.

A Bacillus may encode a polypeptide for treating or preventing a cancer. In a particularly preferred embodiment a construct of the invention may encode a tumour antigen. Examples of tumour associated antigens include, but are not limited to, cancer-testes antigens such as members of the MAGE family (MAGE 1, 2, 3 etc), NY-ESO-1 and SSX-2, differentation antigens such as tyrosinase, gp100, PSA, Her-2 and CEA, mutated self antigens and viral tumour antigens such as E6 and/or E7 from oncogenic HPV types. Further examples of particular tumour antigens include MART-1, Melan-A, p97, beta-HCG, GaINAc, MAGE-1, MAGE-2, MAGE-4, MAGE-12, MUC1, MUC2, MUC3, MUC4, MUC18, CEA, DDC, P1A, EpCam, melanoma antigen gp75, Hker 8, high molecular weight melanoma antigen, K19, Tyr1, Tyr2, members of the pMel 17 gene family, c-Met, PSM (prostate mucin antigen), PSMA (prostate specific membrane antigen), prostate secretary protein, alpha-fetoprotein, CA125, CA19.9, TAG-72, BRCA-1 and BRCA-2 antigen. Examples of particular cancers that the antigen may be derived include those from cancers of the lung, prostate, breast, colon, ovary, testes, bowel, melanoma, a lymphoma and a leukaemia.

The antigen may be an antigen involved in an autoimmune disorder. Thus, the antigen may be an autoantigen.

### Stimuli Detection

The Bacillus of the invention may display a colour change when sporulating or germinating as the spore and vegetative forms of the Bacillus may be a different colour. This means that such Bacilli may be used to detect particular stimuli. Thus, in one preferred instance of the invention, the Bacillus employed displays such a colour change.

Using germination to detect stimuli is typically very quick as a detectable colour change may occur in minutes. In addition, spores can be produced relatively inexpensively and are extremely durable meaning that they have long storage lives, representing further advantages for their use in stimuli detection.

Thus, in one instance, the invention provides a method of detecting a stimulus comprising:

(i) providing spores of a Bacillus which are triggered when the stimulus is present to germinate to give vegetative cells, where the spores and the vegetative cells are a different colour because of differential presence of at least one carotenoid in the spore and vegetative cell forms;

(ii) exposing the spores to the test conditions where it is desired to determine whether the stimulus is present or absent; and

(iii) detecting the presence or absence of the colour change resulting from germination of spores in order to determine the presence or absence of the stimulus.

Any of the Bacilli discussed herein which change colour between their vegetative and spore forms may be employed in such methods.

In one instance, the stimulus itself is a germinant that triggers germination of the spores. For instance, the stimulus may be an environmental stimulus such as light, moisture, pH (in particular low pH) and/or temperature change (particularly temperature shock). Alternatively, the stimulus results in the production of a germinant that triggers germination of the spores.

Examples of germinants include amino acids, nucleosides (e.g. adenosine or inosine), a sugar (e.g. glucose) and/or a metabolite from a different bacterium, particularly a pathogen. Examples of an amino acid which could act as a germinant include L-alanine, L-proline, L-leucine and/or L-valine. In a particularly preferred instance the germinant is an amino acid. Such germinants may also be used in the various methods described herein where it is desired to trigger germination. The conditions the method are carried out under will be appropriate to allow germination if the stimulus is present.

In a preferred instance, the method involves a substrate that is modified in the presence of the stimulus to yield a germinant which triggers germination of the spores. Thus, the stimulus may be the presence of a particular agent that causes the conversion of a substrate, which has no effect on the spores, into a germinant which triggers germination of the spores and hence allows detection of the stimulus. For instance, the stimulus may bring about cleavage of the substrate to yield the germinant. In some instances, the stimulus may trigger a series of events which leads to the generation of a germinant.

In a preferred instance, the stimulus may be the presence of an enzyme capable of modifying the substrate to yield the germinant and in particular a protease or aminopeptidase capable of cleaving the substrate to yield a germinant. In a preferred instance, the substrate may be a dipeptide that may be cleaved to give rise to an amino acid which can act as a germinant. In one instance, the dipeptide may comprise any of the amino acids mentioned herein and in particular a dipeptide, especially of the same amino acid. In a particularly preferred instance, the peptide may be Ala-Ala, the cleavage of which yields Alanine which can act as a germinant.

In a preferred case, the stimulus may be the presence of a particular organism, in particular a microbe and preferably a pathogen. The organism may, for example, release an enzyme which can cleave the substrate and hence trigger germination. The bacteria may release any of the enzymes discussed above which lead to modification of the substrate and hence germination of the spores. The ability to detect microbes may be used to determine if a sample comprises microbes.

The method may be used to test for sterility and/or contamination. The method may be used to detect undesirable agents and, in a preferred instance, to detect biological agents which may be used in warfare or terrorism. Such methods may be used to detect the presence of microbes in biological materials such as, for instance, blood, plasma, serum and tissue samples. The invention may be used for testing for contamination in tissue banks and blood banks. The method may be used to test for microbial contamination in packaged goods such as in pharmaceuticals and drugs. In a preferred instance the invention may be used to test for contamination in liquid samples, such as in pharmaceutical products and in tissue culture. Mycoplasma contamination in tissue culture may be tested for.

The invention may also be used to check sterility or contamination of serum, saline drips, cells culture medium, buffers, water and liquids in general. The method may be used to monitor for sterility during manufacture, such as in drug or food manufacture.

The methods may be used to detect the contamination of sterilized materials. The methods may be employed to detect the organisms in hospitals, schools, government and military establishments.

Examples of microbes which may be detected include any of those mentioned herein and preferably pathogenic Bacillus strains such as Bacillus anthracis and Bacillus cereus, and in particular Bacillus anthracis. MRSA may be detected. Pathogens which may be detected include ETEC (enterotoxigenic E. coli), vibrio cholerae, Mycoplasmas, Pseudomonas aeuroginosa, Corynebacterium diptheriae, salmonella typhi, Shigella flexneri, S. aureus, Streptococcus pyogenes and Neisseria meningitides, Shigella dysenteriae, Entameoba histolytica, Cryptosporidium paruum, Giardie lamblia, pfiesteria, Toxoplasma gondii; and Campylobacter.

In one preferred instance, the germinating spores, or vegetative cells resulting therefrom, may themselves be able to give rise to a stimulus triggering the germination of the other spores present. This can lead to an amplification of the initial trigger making the method highly sensitive. Thus, a very small amount of the stimulus may trigger a detectable colour change. For instance, as little as a 1000 or less, preferably 100 or less, more preferably 25 or less, even more preferably 10 or less or even more preferably the presence of a single microbe may be able to trigger a detectable colour change. In other instance, the germination of spores may not trigger the germination of others. This can allow the method to be made quantitative or semi-quantitative by detecting the intensity of the colour change.

The colour change in the above methods may be detected using any suitable means. For instance, a colour change may be detected by eye, by using spectrophotometry, through the use of a digital camera or any other sensing means that can be used to detect colour change. The detection of colour change may be automated. In some instances the method may be qualitative, semi-quantitative or quantitative. In many embodiments, the higher the level of the stimulus, the more spores germinate, the greater the colour change and hence the method may be quantitative or semi-quantitative. Standards with different amounts/levels of a particular stimulus may be used as controls. In some instances a test sample may be added to the spores and the presence or absence of a colour change detected. In others, the biosensor may be present in situ. The biosensor may preferably give a result in under an hour, preferably under 30 minutes and even more preferably under 15 minutes when exposed to the stimulus.

The invention also provides a biosensor comprising the Bacillus and based on the above discussed colour change resulting from germination. In a particularly preferred instance, the biosensor also comprises a substrate which can be modified by the stimulus to provide a germinant as discussed above. In a further preferred instance, the biosensor comprises the Bacillus provided on a support. The support used in the biosensor according to the invention preferably provides an inert surface that can support a spore and may have a colour which is different to the colour of the spore or of a vegetative cell which might germinate from the spore. The colour of the support is preferably a contrasting colour to the spores of the Bacillus. The support, for instance, may comprise silicon (e.g. a silicon wafer), paper (e.g. filter paper), mica, polystyrene, polyethylene, polycarbonate, or a lipid film (e.g. a phospholipid bi-layer or mono-layer). The biosensor according to the invention may optionally be miniaturised and be provided on an array comprising a plurality of biosensors according to the invention. In some instances, the biosensor may include control regions designed to give a positive or negative test result.

In some instances, the biosensor may be adapted to analyse multiple samples. For instance, the biosensor may be in a format which facilitates automated reading and may be in a multiwell format. In some instances, a sample may have to be added to the biosensor to test it for the presence or absence of the stimuli. In other instances, the biosensor may sense the stimulus without having to add a sample to it. The biosensor may, for example, be left in situ and any colour changes indicates the presence of the stimulus, such as, for instance, the presence of a pathogen.

The invention also provides a kit for detecting a stimulus, the kit comprising:

(i) spores of a Bacillus which are triggered to germinate when the stimulus is present to give vegetative cells, where the spores and the vegetative cells are a different colour because of differential presence of at least one carotenoid in the spore and vegetative cell forms; and

(ii) instructions for how to use the Bacillus to detect the presence of the stimulus.

The invention also provides a kit comprising:

(i) spores of a Bacillus which are triggered to germinate when the stimulus is present to give vegetative cells, where the spores and the vegetative cells are a different colour because of differential presence of at least one carotenoid in the spore and vegetative cell forms of the Bacillus; and

(ii) a substrate which is modified in the presence of the stimulus to give rise to a germinant which triggers germination of at least one of the spores.

The present invention may also be used to assess a method of killing or inactivating spores comprising:

(i) exposing spores of a Bacillus to the test conditions or agent, where the spores of the Bacillus are of a different colour to the vegetative cells of the Bacillus because of differential presence of at least one carotenoid in the spore and vegetative cell forms of the Bacillus; and

(ii) contacting the spores of the Bacillus with a germinant and detecting the presence or absence of the colour change resulting from spore germination in order to determine whether the spores have been killed or inactivated.

Such methods may in particular be used to determine the efficacy of sterilisation and in particular to assess the efficacy of autoclaving. Such methods may be used to determine the efficacy of techniques to kill anthrax spores or other sporulating pathogens which may be used as biological weapons or in bioterrorism.

The invention also provides a method for identifying antibiotics comprising:

(i) providing spores of a Bacillus, where the spores of the Bacillus are a different colour to vegetative cells due to the differential presence of at least one carotenoid in the spore and vegetative cell forms of the Bacillus;

(ii) contacting the spores with a test substance under conditions suitable for germination of the spores; and

(iii) detecting any change in colour, or colour intensity, in order to determine whether the test agent prevents growth of the Bacillus.

The colour change looked for may in some instances be the colour change resulting from germination and subsequent growth of the Bacillus and in some instances the increase in the number of vegetative cells. Prevention of such colour changes indicates that the test agent has antibiotic properties which can then be examined in more detail. Appropriate controls may be run with compounds known to have antibiotic activity and/or compounds known to lack such activity.

Suitable test substances which can be tested in the above assays include combinatorial libraries, defined chemical entities and compounds, peptide and peptide mimetics, and natural product libraries. Typically, organic molecules will be screened, preferably small organic molecules which have a molecular weight of from 50 to 2500 daltons. Candidate products can be biomolecules including, saccharides, fatty acids, steroids, purines, pyrimidines, derivatives, structural analogs or combinations thereof. Candidate agents are obtained from a wide variety of sources including libraries of synthetic or natural compounds. Known pharmacological agents may be subjected to directed or random chemical modifications, such as acylation, alkylation, esterification, amidification, etc. to produce structural analogs. Test substances may be used in an initial screen of, for example, 10 substances per reaction, and the substances of these batches which show activity tested individually.

### Production of Carotenoids, Carotenoid Precursors and Desired Derivatives

The Bacilli discussed herein may be used in the production of desired compounds. In a particularly preferred instance, the Bacilli may be used to produce carotenoids as well as carotenoid precursors and derivatives of carotenoids. The advantages of particular bacterium according to the invention include that they may be used as a natural source of carotenoids having different colours due to the differential presence of at least one carotenoid. Different classes of carotenoids are produced at different stages of bacterial development. The different physical and chemical properties of these compounds confer different colours.

Thus the invention provides method of producing a carotenoid, carotenoid precursor or a derivative of a carotenoid comprising growing vegetatively and/or producing spores of a Bacillus described herein. The method will typically involve harvest of the desired compound from the Bacillus. In some cases, spores or vegetative cells will be broken to provide an extract comprising the desired compound. The desired compound may be further isolated from the other components of the spore and/or vegetative cell.

Carotenoids may be isolated from the Bacilli. The carotenoids may be used in any of the aspects discussed herein in place of the Bacillus. Various techniques may be used to obtain carotenoids from the Bacilli. The carotenoids may be isolated and purified from the culture. In particular, the carotenoids can be extracted using an organic solvent. The Bacillus may first be separated from the culture by conventional techniques such as centrifugation or filtration and then the carotenoids extracted. As a solvent for the extraction, any substance in which the desired compound is soluble can be used. For example, organic solvents such as dichloromethane, benzene, acetone, chloroform, hexane, cyclohexane, methanol, ethanol, isopropanol, carbon disulfide, diethyl ether, acetone may be employed. Examples of particularly preferred solvents include chloroform, acetone, hexane and diethyl ether. The purification can be carried out by conventional chromatography procedures such as absorption, elution, dissolving and the like, alone or preferably in combination.

Thus, in one instance, the invention provides a method of producing a carotenoid, a carotenoid precursor, or a derivative of a carotenoid, which method comprises:
providing a Bacillus of the invention;
causing or allowing growth and/or sporulation of the Bacillus and hence production of the carotenoid; and
extracting the carotenoid from the Bacillus.
   In one instance, the method may comprise inducing sporulation of the vegetative form of the Bacillus and extracting carotenoid from the spore. In another instance, carotenoids or other compounds are extracted from the vegetative cells of the Bacillus.

The carotenoid produced by the bacterium according to the invention is preferably a keto/hydroxyl-carotene derivative (such as hydroxyl-spheriodene, 1-HO-demethylspheroidene, 3,4-dihydrospheroidene and/or 15-cis phytoene) in the vegetative cell. Since the bacterium according to the invention produces cyclic carotenoids having keto and hydroxyl moieties, it has the potential for producing astaxanthin, 4-ketozeaxanthin, echinenone, a hydroxyl-echinenone derivative, phenicoaxanthin, canthaxanthin, zeaxanthin, β-carotene γ-carotene and/or a keto/hydroxy derivative of a mono-cyclic carotenoid and the Bacilli may be used for such a purpose. In addition, a fatty acid ester and/or a glucoside derivative may be produced.

One particular advantage of the invention is that both the Bacilli and carotenoids extracted from them may be acid resistant, particularly gastric acid resistant. This has the advantage that organisms receiving the Bacilli or carotenoids may be able to derive more benefit from them in terms of the amount of carotenoids absorbed or taken up. In one instance, the Bacilli or carotenoids may display resistance to degradation of carotenoids in simulated gastric conditions. For instance, simulated gastric fluid (SGF) may be used. An example of SGF is 1 mglml pepsin dissolved in 0.9% NaCL(pH2) with incubation at 37° C. for one hour. The SGF and assay may be adjusted to mimic the conditions in any of the organisms discussed herein. In one instance, after a one hour incubation at least 25%, preferably at least 50%, even more preferably at least 60%, even more preferably at least 70% and in particular at least 80% of the carotenoids may remain. Any of the assays discussed herein may be employed to measure carotenoid levels.

The Bacilli of the invention may be ones modified to produce particular desired carotenoids and/or other desired metabolites. Carotenoids, taxanes and artemisinin are examples of high-value fine chemicals. These compounds are all isoprenoids and therefore biosynthetically related via the C₅ precursor Isopentenyl diphosphate (IPP). Isoprenoids such as ubiquinone are essential for microbial growth and therefore an organism must possess an isoprenoids biosynthetic pathway. However, the classes of isoprenoids formed by this pathway show extensive metabolic biodiversity. For example, most bacteria do not possess the ability to generate the C₂₀ prenyl precursor geranylgeranyl diphosphate (GGPP). This isoprenoid is the precursor for carotenoids and the anticancer compounds taxanes. Farnesyl diphosphate (C₁₅; FPP) is the precursor for the anti-malaria drug artemisinin. Thus, the Bacilli according to the invention are an important utilizable source of an isoprenoid precursor for both carotenoid and taxane formation.

The Bacilli discussed herein may be used in the production of such compounds and in particular isoprenoids. They may naturally express such compounds, have been genetically modified to produce such compounds and/or have been modified to over-express such compounds. The Bacilli may be used to produce GGPP and/or Farnesyl disphosphate which may be then harvested from the Bacilli. The harvested compounds may then be used to synthesize taxanes or artemisinin. Alternatively, the Bacillus may be able to synthesize such compounds directly which can then be harvested.

The presence of an active endogenous GGPP forming pathway alleviates the necessity to engineer a precursor pathway into the Bacillus, which has previously been found to be necessary for other organisms. In addition, the absence of a highly active sterol pathway in the Bacilli of the invention prevents diversion of carbon into these essential membrane components. For example in order to optimise carotenoid production in yeast, it has been found necessary to down-regulate squalene synthase to divert FPP from the sterol pathway into GGPP and then carotenoids. In addition, the Bacillus forms IPP via the 1-deoxy-D-xylulose-5-phosphate (DXP) pathway, e.g. from pyruvate and glyceraldehyde-3-phosphate.

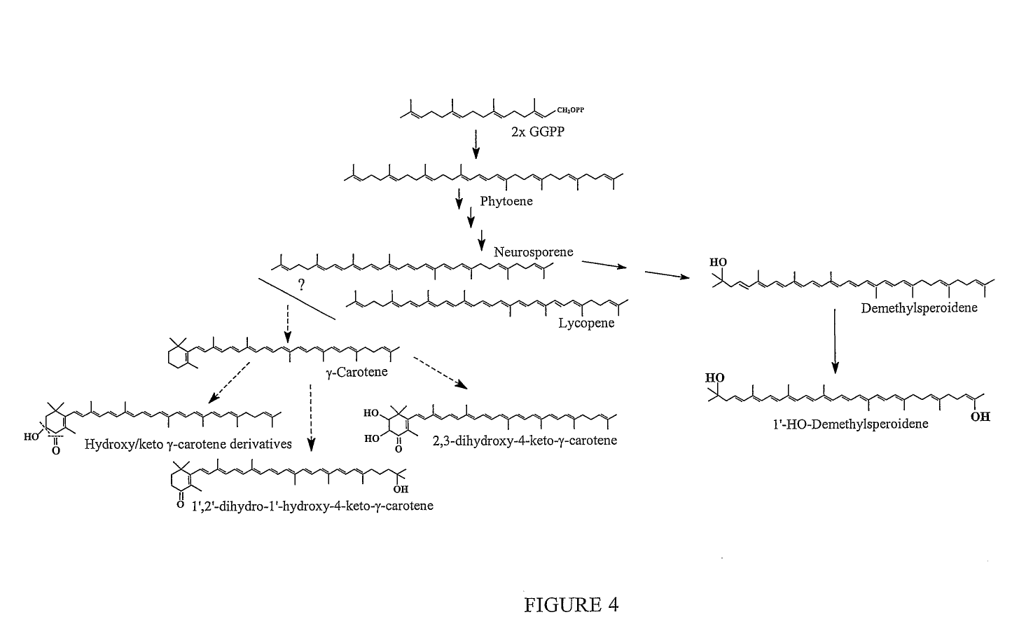

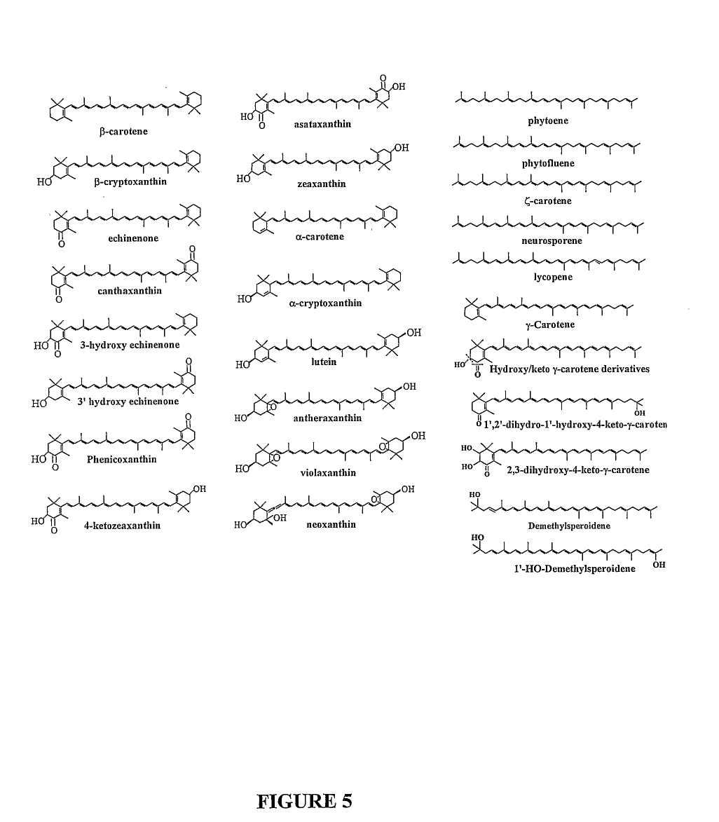

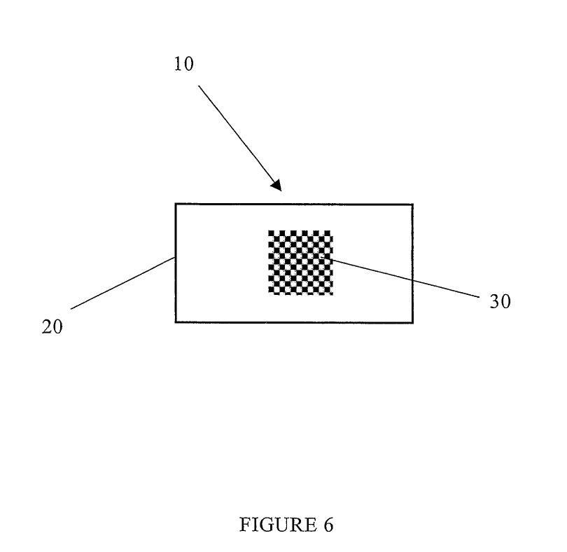

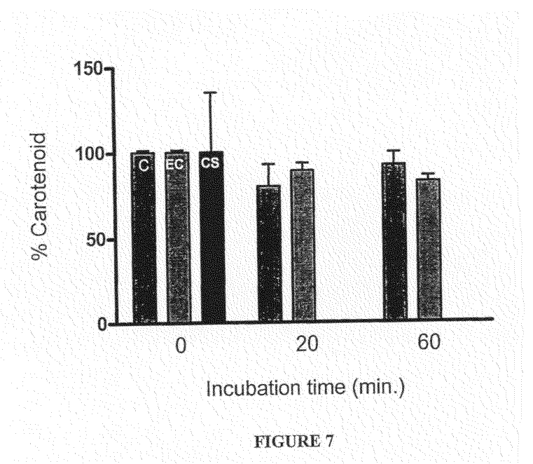

Since the Bacillus expresses one or more of the above products, the metabolic diversity of the products is extended considerably. In addition to these products, esterified and glucoside derivatives of them may also be expressed. The Bacilli may, for instance, be used to produce any of the compounds shown in FIGS. 4 and 5.

In one preferred instance, genes of the mevalonate pathway may be introduced into the Bacillus. For instance, one or more, or in a preferred case all of, the atoB, HMGS, tHMGR, ERG12, ERG8, MVD1, idi and ispA genes, or functional equivalents thereof, may be introduced into the Bacillus. In an especially preferred instance, an amorpha-4,11, diene synthase (ADS) gene may be introduced and in particular in addition to the MVA gene or genes to allow synthesis of amorpha-4,11, diene a valuable precursor of the anti-malarial drug artemisinin. Thus, amorpha-4, 11, diene may be synthesised using the Bacillus of the invention. In a preferred aspect, the pathway engineering to introduce the MVA pathway described in Martin et al (2003) may be employed.

In preferred instances:
an atoB gene may be introduced to allow synthesis of acetyl-CoA (AA-CoA);
an HMGS gene may also be introduced to allow synthesis of hydroxymethylglutaryl-CoA (HMG-CoA);
a tHMGR gene may also be introduced to allow the production of mevalonate;
an ERG12 gene may be introduced to allow synthesis of mevalonate 5-phosphate (Mev-P);
an ERG8 gene may be introduced to allow synthesis of mevalonate pyrophosphate (Mev-PP);
a MVD1 gene may be introduced to allow synthesis of isopentyl pyrophosphate (IPP);
an idi gene may be introduced to allow synthesis of dimethylallyl pyrophosphate (DMAPP);
an ispA gene may be introduced to allow synthesis of OPP; and
an amorpha-4,11, diene synthase (ADS) gene may be introduced to allow synthesis of amorpha-4,11, diene.

Bacillus are provided which have had introduced the genes necessary to produced one of the compounds mentioned above. Thus, by only introducing part of the pathway intermediates in the pathway may be produced. Any of the Bacilli discussed herein can be so modified, particularly one with at least 95% 16S rRNA sequence identity to any of SEQ ID Nos 1 to 6. In one instance, any of the strains HU19, 28, 33, and 36 may have had such modifications introduced into them.

The introduced mevalonate pathway also facilitates the opportunity to generate radiolabelled and stable isotope labelled carotenoids from simple precursors and the invention provides for the production of such radiolabelled and stable isotope labelled carotenoids using the Bacilli discussed herein. Examples of precursors which may be employed include mevalonate and/or acetate being added to the media. There is a need for such compounds as they are not presently available commercially. The invention provides Bacilli comprising such compounds as well as the compounds obtained from the Bacilli.

The invention also provides extracts from the Bacillus. In particular, extracts comprising carotenoids are provided as well as optionally any of the other compounds mentioned herein. Extracts may be whole cell or spore extracts or alternatively may have been further purified to increase the relative amount of the desired compound. In one instance, an extract of all of the carotenoids present in a particular cell or spore is provided. In another instance, the extract comprises one particular carotenoid from the Bacillus. In one instance, a gastric acid resistant carotenoid extract is provided.

### Foods, Food Supplements and Other Compositions

The Bacilli described comprise carotenoids and hence have nutritional value and may also be orally consumed to provide colour in the tissues of animals, particularly those that are in turn going to be used as foods. Thus, the Bacilli discussed herein, and the materials obtained from them, may be used as orally consumed agents and in particular in foods and food supplements. In a preferred instance, the spores of the Bacilli may be orally consumed. In other instance, rather than the intact Bacillus, materials obtained from the Bacillus may be orally consumed and in particular carotenoids may be so consumed. Thus, an extract from the Bacillus may be consumed, isolated carotenoids or a carotenoid rich fraction may be consumed. Such uses of the Bacilli may result in increased yields from organisms such as, for instance, in farmed organisms.

The present invention therefore provides a foodstuff or a food-supplement comprising any of the Bacilli discussed herein or extracts from such strains. The foodstuff, or supplement, may be one for human consumption. In others, they may be for non-humans, for instance in, or as, foods for commercially raised/farmed organisms. In one instance, any of the Bacilli discussed herein, or extracts from them, may be used as food colourants and hence the invention provides a food colourant comprising a Bacillus discussed herein or an extract from such a Bacillus.

A food is typically an edible material composed primarily of one or more of the macronutrients protein, carbohydrate and fat, which is used in the body of an organism to sustain growth, repair damage, aid vital processes or furnish energy. A food may also contain one or more micronutrients such as vitamins or minerals, or additional dietary ingredients such as flavourants and colourants. The term food as used herein also covers a beverage. Examples of foods which the Bacilli may be incorporated include snack bars, cereals, baked goods, dips and spreads, confectionery, probiotic formulations including yoghurts, and frozen confections. Preferred foods include yoghurts, cheeses and other dairy products. Sweets and candy, especially soft jelly type sweets, may incorporate the carotenoids. Examples of beverages include soft beverages, squashes (e.g. orange and lemon), dry drink mixes, nutritional beverages and teas. The beverage may be an alcoholic beverage and, for instance, the Bacilli, or extracts obtained from them, may be used in alcohol-pops to add colour. In one instance, the food may be one that comprises an aquatic animal, or meat therefore, in an alternative instance, the food may be one that does not comprise an aquatic animal or meat or other materials therefrom. In one instance it may be a fermented food. In another it may be a non-fermented food. In one instance, the food is not Jeotgal.

In one preferred embodiment of the invention, the Bacillus, or materials obtained from them, may be fed to non-human animals and in particular such animals from which foods or other materials are harvested, and especially fed to animals that are used as food. Thus, food, food supplements and food additives for such animals may comprise the Bacilli discussed herein, extracts from such Bacilli or have been fed such extracts or Bacilli at some point.

In one preferred instance the animals may be aquatic animals. Examples of fish and other aquatic animals include, for instance, sharks, rays, sturgeon, eels, anchovy, herring, carp, smelt, salmon, trout, hakes, cod, rockfish, bass, drum, mackerel, tuna, butterfish, catfish, flounder and seabream. In one preferred instance, the fish may be a salmon or a trout and in particular may be a salmon. In one instance the fish is a member of the Salmonidae family. The animal may be a mollusc including bivalves, gastropods, cephalopods and chitons such as, for instance, a mussel, a clam, an oyster, a scallop, a snail, a conch, an abalone, a squid and a cuttlefish. They may be fed to crustaceans. Examples of crustaceans include shrimps, prawns, lobsters, red claws, crayfish crabs, Moreton Bay bugs, and marron. Examples of preferred crustaceans include shrimps and in particular Penaeus monodon (black tiger shrimp) or other Penaeus species. In some instances the animal may be a non-aquatic organism and in particular poultry, cattle, swine, and sheep may be fed, especially such farmed animals. Chickens and turkeys may in particular be fed such Bacilli or materials obtained from them. Processed foods made from such organisms are also provided.

In one preferred instance the food or food supplements may be fed to a farmed animal and in particular to a commercially farmed aquatic animal. For instance, such supplements may be fed to farmed trout, salmon or shell-fish and in a preferred instance to farmed shrimps or prawns, especially farmed shrimps, including any of those types mentioned herein. The animals may be non-aquatic farmed animals.

The Bacilli and extracts from them are typically coloured because of the presence of colourants. They may therefore be used as food colours. They may also be fed to animals so that one or more tissues of the animal become a particular colour, including any of those mentioned herein. Preferably spores may be fed for such a purpose or extracts obtained from them. In a preferred case, the Bacilli and extracts provide a method of providing colour to one or more tissues of a non-human animal comprising feeding the Bacillus, or extract, to the animal. In particular, such techniques may be used to provide colour to farmed fish and also to shrimps and prawns. In one instance, the Bacillus is provided not for the purposes of colour, but to simply enhance yield or to do both.

In some cases where the spores of the Bacillus are orally consumed they may have been treated so they cannot germinate. This may mean be done so that the spore is inert and hence therefore preferably colour-stable. In one instance, heat treatment and in particular autoclaving may be utilised to prevent the spores from germinating. In other instances, the spores may still be able to germinate. In some instances, the purpose of feeding the Bacillus to the organism may alternatively or additionally be, to increase yields. Thus, for instance, average weight and/or weight increases may be greater when comparing the organism fed the Bacillus or extract in comparison to a control that has not been.

The invention also provides a probiotic comprising a Bacillus discussed herein. A probiotic is typically a live bacterial supplement which can enhance the intestinal flora. In some cases probiotics may be given, for instance, after illness or antibiotic treatment to help restore the normal microflora of the animal. Such probiotics may be given to any of the animals discussed herein and in particular to humans, but also to farm and domestic animals and to aquatic organisms. In a preferred instance, the Bacillus is used in spore form for such probiotics. The probiotic may additionally comprise one or more acceptable excipients. An acceptable excipient is an excipient which is suitable to be ingested by an animal and in particular a human. The probiotic may also have various flavourings and be in a form suitable for oral consumption.

The Bacilli of the invention and extracts from them, and in particular carotenoids obtained from them, may be used in the production of pharmaceutical compositions. Thus, the invention provides a pharmaceutical composition comprising a Bacillus of the invention, or a carotenoid extract from such a Bacillus, and a pharmaceutically acceptable excipient or carrier. Carotenoids can act as anti-oxidants and hence the compositions may be used to treat or prevent a variety of conditions.

The Bacilli and extracts from the Bacilli may be used in the manufacture of medicaments to treat or prevent cancer, heart disease, atherosclerosis, cataracts, macular degeneration in the eye, stroke, dementia, Alzheimer's, osteoporosis, chronic fatigue syndrome, and male infertility. They may be used in the manufacture of medicaments for treating skin wrinkles or other characteristics of aging. They may be used to reduce the risk, or delay the onset of, or for treating diabetes. In some instances, they may be used to enhance the effects of chemotherapy.

In one preferred instance, the antioxidant provided through the invention is beta carotene which may, in particular, be used to prevent, reduce the risk of, or delay onset of heart disease. In another preferred instance, the invention may be used to provide beta-carotene, Vitamin E and/or Vitamin C and in particular to decrease susceptibility of LDL (low density lipoprotein) to oxidation. The compositions of the invention may therefore be used to prevent, reduce the risk of, or delay onset of strokes and myocardial infarctions. In a particularly preferred embodiment, the Bacilli and extracts from them may be used in the prevention or treatment of cancer, heart disease and cataracts. Examples of types of cancers which the invention may be applied to include lung cancer, breast cancer, prostate cancer and colorectal cancer, in particular lung and prostate cancer.

The present invention also provides for the use of a Bacillus of the invention, or an extract from such a Bacillus in the manufacture of a medicament, food, food supplement, probiotic, nutraceutical or dietary supplement for improving any of the conditions mentioned herein.

In one embodiment, because the Bacilli and the extracted carotenoids from them may be gastric resistant lower doses may be employed than conventionally in order to give the same benefit, for instance, a half or less, or a quarter or less.

The invention further provides an edible composition which comprises an edible carrier and a Bacillus of the invention or an extract from such a Bacillus, in an amount effective to improve or ameliorate any of the conditions mentioned herein in a subject by whom the composition is consumed and in particular a human subject. The composition is preferably a food product, food-supplement, a dietary supplement, a probiotic, a nutraceutical or a food additive.

A nutraceutical is a food ingredient, food supplement or food product which is considered to provide a medical or health benefit, including the prevention and treatment of disease. In general a nutraceutical is specifically adapted to confer a particular health benefit on the consumer. A functional food is a food that is typically marketed as providing a health benefit beyond that of supplying pure nutrition to the consumer. A functional food typically incorporates an ingredient which confers a specific medical or physiological benefit other than a nutritional effect. A functional food typically carries a health claim on the packaging.

The Bacillus, or extracts therefrom, may also be employed as a colourant or as a dye. In one instance, they may be employed in cosmetics. Thus, the invention provides a cosmetic comprising a Bacillus or an extract therefrom. Examples of cosmetic include lipsticks, mascara, eyeliner, foundation and tanning compositions. The Bacillus, or extracts therefrom, may also be used to help protect against the sun. Thus the invention provides a sun-tan lotion or sun-block comprising them as well as a composition for oral consumption for preventing, reducing or ameliorating sun-burn. Extracts from the Bacillus may be used as dyes for fabrics and other materials as well as food-dyes.

### Formulations

The various compositions of the invention may be in a variety of forms. The compositions may be, for instance, in the form of a tablet, capsule or a powder. When the composition is in the form of a powder, it may preferably be provided in an air-tight container such as a sachet or bottle.

Examples of excipients which may be present in the various compositions of the invention include a diluent (e.g. a starch or cellulose derivative, a sugar derivative such as sucrose, lactose or dextrose), a stabilizer (e.g. a hygroscopic component such as silica or maltodextrin), a binder, buffer (e.g. a phosphate buffer), a lubricant (e.g. magnesium stearate), coating agent, preservative, emulsifier, dye, flavouring, and/or suspension agent. Suitable excipients are well known to a person of skill in the art.

The various products of the invention may comprise a carrier or excipient which may be a solvent, dispersion medium, coating, isotonic or absorption delaying agent, sweetener or the like. These include any and all solvents, dispersion media, coatings, isotonic and absorption delaying agents, sweeteners and the like. Suitable carriers may be prepared from a wide range of materials including, but not limited to, diluents, binders and adhesives, lubricants, disintegrants, colouring agents, bulking agents, flavouring agents, sweetening agents and miscellaneous materials such as buffers and adsorbents that may be needed in order to prepare a particular dosage form.

For example, the solid oral forms may contain, together with the active compound, diluents such as lactose, dextrose, saccharose, cellulose, corn starch or potato starch; lubricants such as silica, talc, stearic acid, magnesium or calcium stearate and/or polyethylene glycols; binding agents such as starches, arabic gums, gelatin, methylcellulose, carboxymethylcellulose, or polyvinyl pyrrolidone; disintegrating agents such as starch, alginic acid, alginates or sodium starch glycolate; effervescing mixtures; dyestuffs, sweeteners; wetting agents such as lecithin, polysorbates, lauryl sulphates. Such preparations may be manufactured in known manners, for example by means of mixing, granulating, tabletting, sugar coating, or film-coating processes.

Liquid dispersions for oral administration may be syrups, emulsions and suspensions. The syrups may contain as carrier, for example, saccharose or saccharose with glycerol and/or mannitol and/or sorbitol. In particular, a syrup for diabetic patients can contain as carriers only products, for example sorbitol, which do not metabolise to glucose or which only metabolise a very small amount to glucose. The suspensions and the emulsions may contain as carrier, for example, a natural gum, agar, sodium alginate, pectin, methylcellulose, carboxymethylcellulose or polyvinyl alcohol. As carotenoids are lipid soluble, appropriate formulation will be employed to take that into account.

Suitable types of formulation are fully described in Remington's Pharmaceutical Sciences, Mack Publishing Company, Eastern Pennsylvania, 17^{th} Ed. 1985, the disclosure of which is included herein of its entirety by way of reference.

In one instance, the compositions of the invention are administered to achieve a daily intake of between about 10⁴ to about 10¹⁴ colony forming units (CFU) of the Bacillus according to the invention. In one instance the composition is administered to achieve a daily intake of between about 10⁶ to 10¹² CFU of the Bacillus.

The invention will now be illustrated with reference to the following Examples which are not intended to limit the scope of the invention claimed.

### Example 1

Vegetative cell growth was made on LB agar and sporulation on DSM (Difco Medium) agar (Nicholson and Setlow, 1990). To prepare large quantities of spores free from vegetative cells sporulation was made in DSM liquid medium using the exhaustion method as outlined elsewhere (Nicholson and Setlow, 1990). In this method sporulation was allowed to proceed for 24 h at 37° C. before removal of contaminating vegetative cells by lysozyme treatment. Vegetative cells were prepared by growth of bacteria in LB medium (37° C.) until cultures reached an OD₆₀₀ nm of approximately 2.0.

Heat-resistant spores present in freshly voided human feces (that retained their pigmentation) were isolated. On average, spore counts found in feces were in the range of 10⁴ cfu/g. Using this approach, six yellow-orange pigmented colonies were readily discernable on sporulation agar plates. These isolates were labelled Bacillus A to Bacillus F. Basic characteristics are shown in Table 1.

Resistance to arsenate and aresenite was determined as described (Suresh et al., 2004). Tolerance to NaCl was made on LB agar containing NaCl. Sporulation efficiency was determined by growth and sporulation on DSM agar (3 days at 37° C.) followed by measurement of heat-resistant (65° C. 1 h) cfu/ml vs. unheated cfu/ml. A non-pigmented spore former, Bacillus subtilis strain, PY79 (Youngman et al., 1984), was used as a control. Anaerobic growth was determined using sealed containers and the Oxoid Gas Pak system.

TABLE 1

Bacillus species^{a}

| | 1 | 2 | 3 | 4 |
|---|---|---|---|---|
| Colour | YO | YO | YO | YO |
| Spore position | S | S | S | S |
| Swollen sporangium | + | + | + | + |
| Starch hydrolysis | + | + | + | + |
| Growth at 10° C. | + | W | W | ND |
| 15° C. | + | + | + | + |
| 20° C. | + | + | + | + |
| 40° C. | + | + | + | − |
| 45° C. | + | − | − | − |
| 50° C. | − | − | − | − |
| Anaerobic | − | − | − | − |
| Growth in presence of | | | | |
| Arsenate Na₂HAsO₄ | | | | |
| 5 mM | + | + | + | + |
| 9 mM | + | + | + | + |
| 20 mM | + | + | + | + |
| Arsenite As₂O₃ | | | | |
| 1 mM | − | − | − | + |
| 3 mM | − | − | − | + |
| Max NaCl tolerance | 8% | 8% | 8% | 2% |
| Sporulation efficiency^{b} | 60.8% | 1.1% | 3.5% | ND |
| Motile | − | − | − | − |

Key to Table 1:
^{a}Bacillus species
1, Bacillus A, Bacillus B, Bacillus C
2, Bacillus D
3, Bacillus E, Bacillus F
4, Bacillus indicus sp. nov. Sd/3^{T} (Suresh et al., 2004)
Key: ND, no data; W, weak; Cr, cream; O, orange; W, white; Y, yellow; C, central; S, subterminal; T, terminal
^{b}sporulation determined on DSM agar plates after 3 days at 37° C.

All six pigmented colonies produced spherical spores within swollen sporangia. These isolates were further distinguished by being non-motile, able to hydrolyze starch (amylase positive) and failing to grow anerobically. Strains of at least one yellow pigmented Bacillus species, B. indicus, are arsenic resistant (Suresh et al., 2004) so the isolates were tested for tolerance to both arsenate and arsenite. It was found that all six isolates were able to tolerate up to 20 mM arsenate but not arsenite. All six isolates were able to grow in up to 8% NaCl. Three isolates, Bacillus D, Bacillus E and Bacillus F exhibited poor sporulation efficiencies using the exhaustion method for sporulation in DSM medium.

### Example 2

The pigmentation of the isolated spore formers was investigated. Colonies were isolated on their ability to produce pigmented colonies.

Strains HU 13, HU 28, HU 33 and the control strain PY 79 were compared When grown on LB agar colonies of the HU strains initially were yellow after overnight incubation at 37° C. As incubation was continued the colonies of the HU strains gradually assumed an orange hue. The control strain showed the usual cream-grey appearance of Bacillus colonies.

By contrast, sporulation on DSM agar plates produced colonies that were orange. To determine whether the orange colour was specific to spore formation we made cultures of spores grown by exhaustion in DSM medium and ensured that there were no residual vegetative cells using an established protocol of treatment with lysozyme followed by extensive washing. Similarly, cultures of vegetative cells were made using incubation in LB medium until the culture reached an OD₆₀₀ of 2.0. Cultures prepared in this way would be free of any spores. In both cases, spores and vegetative cells were lyophilized and the difference in pigment was clearly distinguished following desiccation with vegetative cells being yellow and spores orange for the HU strains.

### Example 3

Phylogenetic analysis of the strains isolated in Example 1 was performed. To assign strains to bacterial species we sequenced the entire 16S rRNA gene (rrnE) from cells taken from each colony type in a manner as described previously (Hoa et al., 2000). The sequence ID numbers of the 16S rRNA gene of each isolate is given in Table 2:

TABLE 2

| Isolate | Seq ID No. |
|---|---|
| A (HU 13) | 1 |
| B (HU 16) | 2 |
| C (HU 19) | 3 |
| D (HU 28) | 4 |
| E (HU 33) | 5 |
| F (HU 36) | 6 |

The 1,400 bp amplicon was then sequenced and analyzed using BLAST (http://www.ncbi.nlm.nih.gov/) to find the nearest matching species. The sequences were then aligned by ClustalW programme (http://align.genome.jp/) and percentage of similarity recorded.

Neighbour-joining trees are shown in FIG. 1. All were closely related to Bacillus catenulatus, B. indicus, B. jeogtgali and B. cibi.

### Example 4

Carotenoids were extracted from the strains isolated in Example 1. As a control the PY79 strain was used. The PY 79 strain is a laboratory strain of Bacillus subtilis referred derived from the 168-type strain. 168 is a type-strain or reference strain which is a is a wild type non-mutant strain (Youngman et al., 1984).

Bacterial biomass was lyophilized to complete dryness (3 days). The lyophilized material was ground into a homogenous powder using a mortar and pestle. Typically carotenoids and other isoprenoids were extracted from 30 mg of ground material using chloroform. In brief, methanol (250 μl) was added to the dried powder and mixed, then 500 μl of chloroform (500 μl; Anlar) added. The suspension was incubated on ice for 20 min. To the suspension water (250 μl) was added and vortexed. In order to form a partition the suspension was centrifuged for 3 min at 12,000 g. The organic hypophase was removed and the aqueous hyperphase re-extracted twice. The organic extracts were pooled and reduced to complete dryness under a stream of nitrogen gas. The dried extracts can be stored at this stage at −20° C. under nitrogen.

### Example 5

The carotenoids extracted in Example 4 were analysed. The component carotenoids were subsequently separated and analyzed using Waters Alliance 2600S HPLC with on-line PDA detection following the procedure described in (Fraser et al., 2000). The dried extracts were re-dissolved in ethyl acetate (HiperSolv) 50 μl, and then centrifuged for 3 min at 12,000 g to remove any particulate material. Separation of isoprenoids was performed using a RP C₃₀ 5 m column (250×4.6 mm) coupled to a 20×4.6 mm C₃₀ guard column (YMC Inc., Wilmington, N.C., USA) operating at a constant temperature of 25° C. Carotenoids were eluted from the column with a gradient of 95% (A)-methanol, 5% (B)-20% aqueous methanol containing 0.2% (w/v) ammonium acetate for 12 min, a step to 80% (A), 5% (B) and 15% (C)— tert-butyl methyl ether at 12 min, followed by a linear gradient to 30% (A), 5% (B) and 65% (C) by 30 min. The column was returned to the initial conditions and equilibrated over 30 min. A flow rate of 1 ml/min was employed and the eluate monitored continuously with a diode array detector between 200 and 600 nm. Identification was performed on the basis of co-chromatography and spectral comparison was authentic standards. Where authentic standards were not available, correlation to reference spectral characteristics were carried out and relative polarities deduced from chromatographic behaviour. For quantification dose-response curves for β-carotene (standard coloured carotenoid) were prepared. Ubiquinone was also identified by co-chromatography and spectral comparison was authentic standards and dose response curves prepared for quantification. All solvents were purchased from VWR Poole, UK.

### Results

All six yellow-orange pigmented isolates were grown in LB medium and screened to reveal the presence of colored carotenoid pigment. The pigmentation was released from the freeze-dried cells upon the addition of chloroform but not methanol. Therefore the pigment was hydrophobic in nature akin to the physical properties of carotenoids. Crude organic extracts were screened by HPLC-PDA without pre-fractionation using an unbiased HPLC separation that facilitates separation and identification of both polar and non-polar carotenoids. The profiles recorded at 250-600 nm of all isolates were found to be similar (data not shown). The predominant peaks at 450 nm showed characteristic signature carotenoid spectra.

TABLE 3
Preliminary screening of yellow-orange pigmented isolates

| | | | Carotenoid content |
|---|---|---|---|
| Form | Isolate | Carotenoid^{a} | (area × 10³/mg culture) |
| Vegetative Cell | A (HU13) | ODMS | 39 |
| | B (HU16) | ODMS | 31 |
| | C (HU19) | ODMS | 152 |
| | D (HU28) | ODMS | 122 |
| | E (HU33) | ODMS | 42 |
| | F (HU36) | ODMS | 143 |

^{a}ODMS, Hydroxy-demethylspheroidene

Those isolates containing the highest level of pigment (e.g. C and F; Table 3) were subjected to further detailed analysis. Accordingly, pure cultures of either vegetative cells or spores as described above were prepared together with B. subtilis strain PY79 that served as a non-pigmented control Bacillus species.

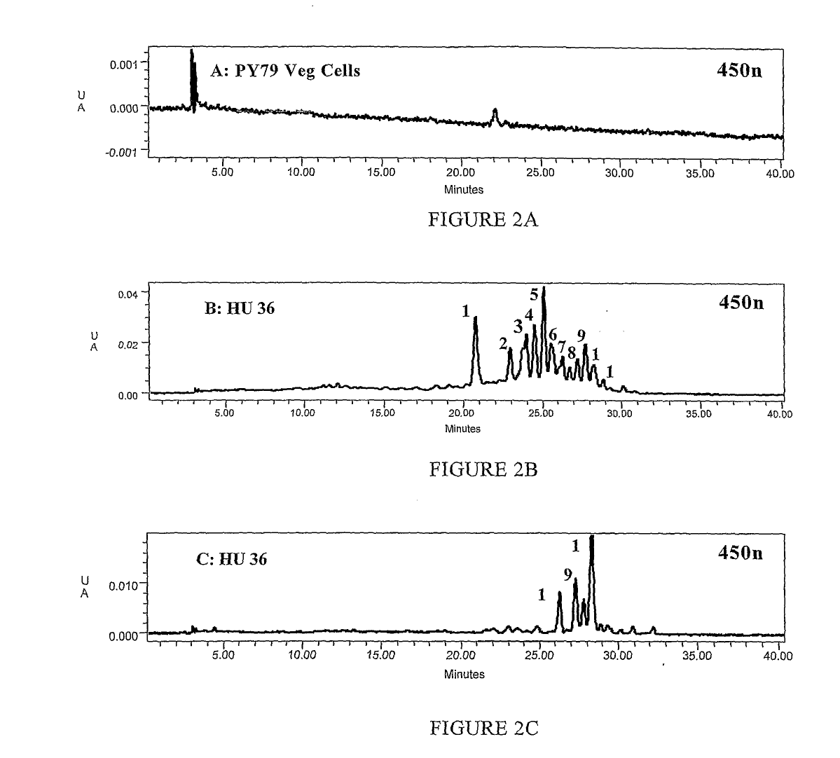

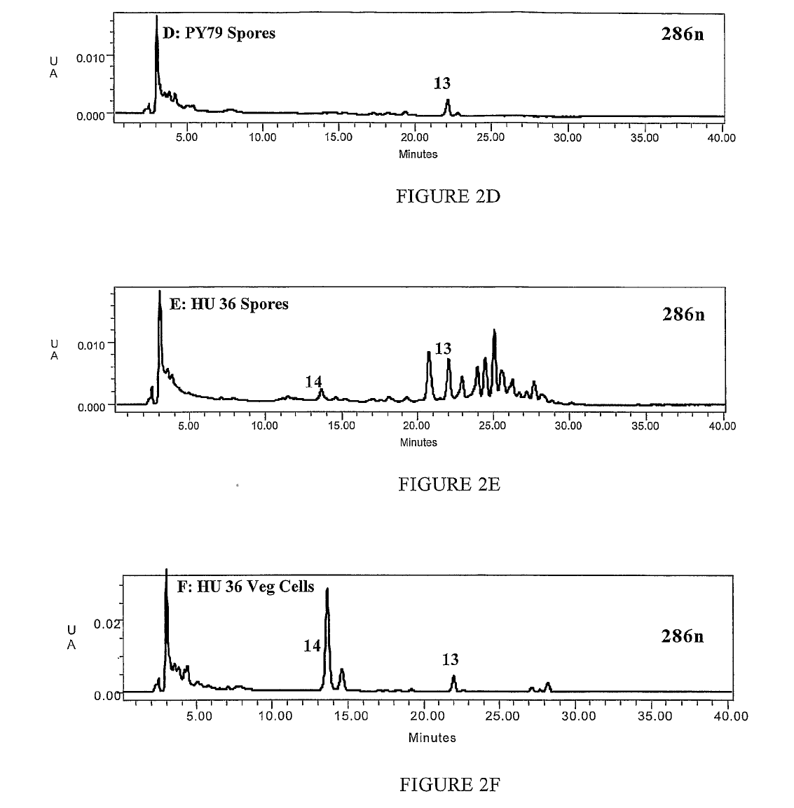

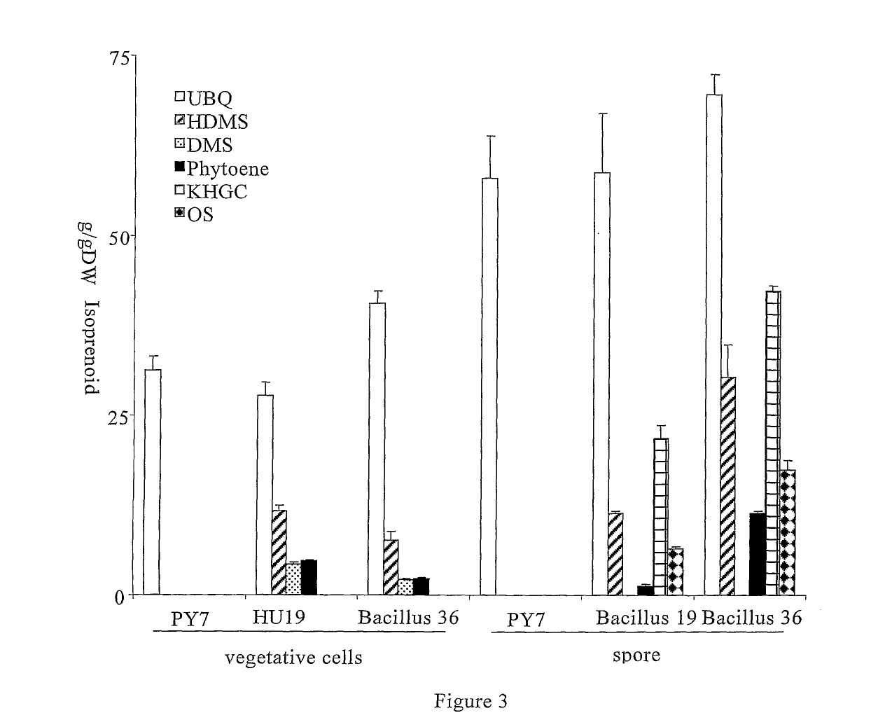

FIG. 2 illustrates the HPLC profiles of the carotenoids found in the isolate F (spores and vegetative cells, FIGS. 2B and 2C respectively) compared to the control PY79 (FIG. 3A). The presence of coloured carotenoids is recorded at 450 nm (FIGS. 2A to 2C), while colourless carotenoids and ubiquinone are displayed in FIGS. 2D to 2F. These profiles are characteristic for all isolates analyzed. No chromatographic components indicating the presence of coloured or colour carotenoids were observed in vegetative cells or spores of the PY79 strain. By contrast, extracts prepared from spores exhibited the presence of at least eleven chromatographic components showing characteristic coloured carotenoids (FIG. 2B). Extracts prepared from vegetative cells contained three predominant coloured carotenoids (FIG. 2C). The carotenoids predominant in vegetative cells possessed spectral maxima at 453.6 nm, the persistence in the spectra suggested that the carotenoid was acyclic in nature. Using authentic standards described in (Badenhop et al., 2003), HPLC peaks 8-11 were identified as 1-HO demethylspheroidene (ODMS), (Table 4). The separation of multiple chromatographic peaks with identical spectra and similar retention times is likely to be due to different geometric isomers. The separation of carotenoid isomers being a common features of the C₃₀ separation stationary phase. Although, a suitable authentic standard was not readily available comparison with reference spectra (Britton et al., 2003) and the relative retention times suggest the identity of HPLC peak 12 to be 3,4-dihydrospheroidene (DHS), (Table 4). From the isomers detected, the carotenoids appear to be predominately in the all-trans configuration and S,S′ stereo-isomer configuration.

TABLE 4
Carotenoid identification based on co-chromatographic and comparative
spectral properties with authentic standards and reference data.

| HPLC peak | | Ret. | | |
|---|---|---|---|---|
| No.^{a} | UV/Vis | time | Carotenoid | Reference Spectra |
| 1 | 465.7, 493.5 | 20.756 | NA | Keto/hydroxy--carotene derivatives, (Britton et al., |
| | | | | 2003) |
| 2 | 468.1 | 22.70 | NA | Keto/hydroxy--carotene derivatives, (Britton et al., |
| | | | | 2003) |
| 3 | 428.2, 453.6, | 23.48 | NA | Hydroxy-spheriodene, (Britton et al., 2003) |
| | 485.0 | | | (Badenhop et al., 2003) |
| 4 | 466.9, 494.7, | 24.25 | NA | Keto/hydroxy--carotene derivatives, (Britton et al., |
| | | | | 2003) |
| 5 | 468.4, 494.7 | 24.85 | NA | Keto/hydroxy--carotene derivatives, (Britton et al., |
| | | | | 2003) |
| 6 | 466.9, 494.7 | 25.33 | NA | Keto/hydroxy--carotene derivatives, (Britton et al, |
| | | | | 2003) |
| 7 | 468.1, 494.5 | 26.03 | NA | Keto/hydroxy--carotene derivatives, (Britton et al., |
| | | | | 2003) |
| 8 | 429.5, 453.6, | 26.98 | 1-HO- | 1-HO-demethylspheroidene, (Badenhop et al., |
| | 486.2 | | demethylspheroidene | 2003) |
| 9 | 428.2, 453.6, | 27.28 | 1-HO- | 1-HO-demethylspheroidene, (Badenhop et al., |
| | 483.8 | | demethylspheroidene | 2003) |
| 10 | 429, 454.8, 486.2 | 28.31 | 1-HO- | 1-HO-demethylspheroidene, (Badenhop et al., |
| | | | demethylspheroidene | 2003) |
| 11 | 428.7, 454.8, | 28.60 | 1-HO- | 1-HO-demethylspheroidene, (Badenhop et al., |
| | 485.0 | | demethylspheroidene | 2003) |
| 12 | 414, 438, 468.0 | 26.29 | NA | 3,4-dihydrospheroidene, (Britton et al., 2003) |
| 13 | 269.8, 330.5 | 21.88 | Ubiquinone | NA |
| 14a | 286.4 | 13.56 | phytoene | 15-cis phytoene, (Britton et al., 2003) |
| 14b | 286.4 | 14.47 | phytoene | All-trans phytoene, (Britton et al., 2003) |

^{a}corresponding to the numbered peaks in FIG. 2;
NA—not available

The presence of ODMS was also found in vegetative cells. However, additional coloured carotenoids were observed in spore extracts. These carotenoids were relatively more polar in their nature than ODMS. HPLC peaks 1 and 3-7 (FIG. 2B) all exhibited similar chromatographic and spectral properties and thus were structurally related (Table 4). Their maximum wavelength (λ) of the carotenoids ranged from 465.7 to 468.4 nm. Thus a shift in their λmax was observed with the carotenoids isolated from spores. These shifts will result in a colour change from those carotenoids determined in vegetative cells. Such an alteration in colour (e.g. yellow to orange) was clearly visible when comparing vegetative and spore derived tissue. Besides increases in the maxima, other features of the spore-derived carotenoids included the disappearance of spectral persistence with a more bell shaped spectra. Inflexions within the spectra were however still observable. Collectively these features indicate structurally the likely presence of a mom-cyclic end group as well as keto and/or perhaps hydroxy moieties. Comparison with reference spectra also matched the identity of carotenoids to Keto/hydroxyl derivatives of γ-carotene (Table 4); (Britton et al., 2003).

Recording of spectra on-line from 280 to 600 nm enabled searching for other essential pathway carotenoids such as ζ-carotene, phytofluene and phytoene. The presence of ζ-carotene or phytofluene was not determined. At 286 nm components of the chromatogram were observed that matched typical spectra exhibited by authentic phytoene (FIGS. 2E and 2F). The earlier retention time suggested that the phytoene determined in the vegetative and spore extracts was probably not 15-cis or all-trans in its geometric configuration (Table 4). The isoprenoid ubiquinone was found in all samples.

Besides the presence of different carotenoids in vegetative cells and spores, quantitative determination revealed a greater carotenoid content in spores as was the ubiquinone content (FIG. 3). The F isolate also exhibited higher levels compared to C.

### Discussion of the Results

Using a combination of HPLC analysis and UV/VIS spectral data we have ascertained that the pigmentation in these Bacillus isolates is due to the presence of carotenoids. Based on the physical characteristics of the carotenoids determined in this study and existing resources available we have assigned the predominant carotenoid species in vegetative cells as ζ-HO-demethylspheroidene and in spores to keto and/or hydroxy-β-carotene derivatives. Thus, there is a quantitative and qualitative difference in end-product carotenoids formed during different developmental stages. From the identity of the end product and intermediate carotenoids determined putative biosynthetic pathways present in vegetative cells and spores can be predicted (FIG. 4). Both vegetative cells and spores appear to have the ability to form neurosporene. Therefore, two GGPP molecules are condensed to form phytoene. This C₄₀ hydrocarbon skeleton with a chromophore of three conjugated double bonds is then subjected to three sequential desaturations at positions 11,12;12′,13′ and 7,8 yielding neurosporene which possess nine conjugated double bonds. In vegetative cells this acyclic carotene can be further methylated, hydroxylated and desaturated. During spore formation it would appear that a mon-cyclisation of an acyclic precursor occurs, to which keto and hydroxy moieties can be incorporated.

### Example 6

A probiotic powder composition according to the invention was prepared as follows and then used to fill 1000 gelatin capsules.

The following ingredients were dry mixed to prepare the probiotic powder composition: 500×10³ CFU of a spore prepared according to Example 1, 200 g of hydroxypropyl methylcellulose, 16 g of stearic acid and 16 g of silica.

### Example 7

A probiotic powder composition according to the invention was prepared as follows and then used to make 1000 tablets using a rotary tablet press.

The following ingredients were dry mixed to prepare the probiotic powder composition: 500×10³ CFU of a spore prepared according to Example 1, 200 g of maltodextrin, 16 g of stearic acid and 16 g of silica.

### Example 8

### Efficacy Trial: A Double Blind Study of HU36 on Penaeus vannamei

### Protocol

Four breeding tanks, each containing 2 m³ of water, were used to house Penaeus vannamei. Each tank housed 35 shrimps. Two tanks were coded and shrimps aged 45 days (5-6 g/shrimp) were fed with “Tomboy™” a commercial shrimp food for one week before the trial commenced. Two tanks were then fed with control or test feed for 30 days. Feeding was three times per day totalling 20 g feed per day. Shrimps were monitored daily. Shrimp weights were measured at day −1 and day 31.

### Materials and Methods

Shrimp tanks held 2 m³ of water, pH 8-8.5, salinity of 2-35% NaCl. The temperature of the water was monitored daily and ranged between 26-33° C. and cleaned using filters containing sand, ground coral and charcoal).

Control feed was “Tomboy™”. Test feed was “Tomboy™” containing HU36 spores at 1×10⁷ spores/g of feed. “Tomboy™” feed was purchased from Ho Chi Minh City.

### Results

TABLE 5

| | Shrimp weights (g) | | | | | | | |
|---|---|---|---|---|---|---|---|---|
| | Day 0 | | | Day 30 | | | | |
| | No. | Total | Av, | No. | Total | Av. | SWG/ | Aver |
| | shrimps | wt | wt/sh. | shrimps | wt | wt/sh. | sh (g) | SWG (g) |
| Cont | 35 | 195 | 5.57 | 34 | 337 | 9.9 | 4.33 | 4.08 |
| 1 | | | | | | | | |
| Cont | 35 | 205 | 5.86 | 35 | 339 | 9.7 | 3.84 | |
| 2 | | | | | | | | |
| Test 1 | 35 | 200 | 5.71 | 35 | 435 | 12.42 | 6.71 | 6.6 |
| Test 2 | 35 | 210 | 6.00 | 34 | 425 | 12.50 | 6.5 | |

### CONCLUSIONS

Average shrimp weight gain (SWG) in the probiotic treated samples comprising HU36 was 162% greater than the controls. Thus, the use of the Bacilli of the invention can substantially enhance yields in aquaculture.

### Example 9

### Gastric Stability of Carotenoids Versus Spore Carotenoids

The rationale of the experiment is that carotenoid supplements are believed to be extremely labile to the gastric juices found in the stomach. Accordingly, there is a need for acid resistant carotenoid preparations for use in supplements.

The stability of total carotenoids found in HU36 spores was measured using extraction and HPLC quantification as previously described for HU36 in earlier Examples.
C: HU36 was incubated with simulated gastric fluid (SGF) for 60 minutes and carotenoid content measured (SGF=1 mg/ml pepsin dissolved in 0.9% NaCl (pH 2) and incubations at 37° C.).
EC: Carotenoids were first extracted from HU36 spores and then incubated in SGF for 60 minutes.
CS: a beta-carotene standard was incubated for 60 min in SGF.

The minimum time for transit through the human stomach is 20 minutes, but the maximum time can reach 45-50 minutes depending on food intake and physiology etc.

The results obtained are shown in FIG. 7. In each case the graph shows carotenoid content versus the measurement at 0 minutes. The carotenoid standard was almost totally degraded by 20 minutes with less than 1% remaining. By contrast, carotenoids present on the spore or spore-extracted carotenoids were essentially stable with at most 20% degradation.

Therefore, the spore carotenoid provides a gastric-resistant source of carotenoid and a carotenoid that is acid resistant which is superior to carotenoid supplements presently available.

### Example 10

### Introduction

The increasing interest in the use of bacteria as functional foods has prompted a number of organisations to consider the safety of probiotic bacteria and guidelines for their use (FAO/WHO, 2002; Sanders, 2003). In the USA, bacteria considered safe for human consumption are awarded GRAS status (“Generally Regarded As Safe”) by the Food and Drug Administration. In the EU a similar system is now under consideration referred to as QPS (“Qualified Presumption of Safety”) and whose aims are to harmonise the safety assessment of microorganisms throughout the food chain (SCAN, 2003a). Typically, lactobacilli and bifidobacteria that are the most common type of probiotic and are used as in a wide variety of fermented dairy products and as probiotic supplement. Despite this usage even these bacteria have been implicated in a number of infections (Borrielo et al., 2003; FAO/WHO, 2002).

Less well known than the lactobacilli and bifidobacteria are probiotics that contain members of the genus Bacillus (Hong et al., 2005; Sanders et al., 2003). A number of species are currently in use including Bacillus clausii, Bacillus subtilis, Bacillus pumilus as well as Bacillus cereus. The advantage of the use of spore forming bacteria is that the spore used enabling storage of the product at room temperature as well as superior resistance to gastric juices. Members of the Bacillus genus are generally considered soil organisms since, of course, they are readily found here. However, they are also found in the gastrointestinal tracts (GIT) of many animals and insects (Hong et al., 2005; Nicholson, 2002). Recent evidence has shown that spores of a laboratory strain of Bacillus subtilis can germinate, replicate and then re-sporulate within the murine GIT (Casula and Cutting, 2002; Hoa et al., 2001; Tam et al., 2006). Two Bacillus pathogens, Bacillus anthracis and Bacillus cereus, are of course, known intestinal pathogens and can germinate and replicate in the GIT (Jensen et al., 2002; Jensen et al., 2003; Mock and Fouet, 2001) so it seems highly probable that most, if not all members of the Bacillus genus when consumed orally as spores can germinate and then grow in the GIT. If these live cells can re-sporulate as has been shown to occur with B. subtilis (Tam et al., 2006) then the shedding of spores in the faeces in the faeces of animals and insects over many years may account for the apparently large numbers of spores present in soil.

A number of reports have referred to Bacillus spp. being linked to infections. The incidence is low and in many cases misdiagnosis can account for these (de Boer and Diderichsen, 1991; Logan, 2004; Osipova et al., 1998; Salminen et al., 1998; Sanders et al., 2003). A number of European regulatory bodies have commented on the safety of Bacillus for human use as well as for their use in animal feeds (anon., 2004; SCAN, 2000) with the emphasis being on B. cereus.

The Food and Agriculture Organization (FAO) of the United Nations and the WHO have provided guidelines for the evaluation of probiotics in food (FAO/WHO, 2002) and similar guidelines are now being proposed within the EU (SCAN, 2003a). Regarding safety the most important FAO/WHO guidelines include determination of antibiotic resistance profiles, characterisation of virulence factors and in vitro and in vivo safety testing.

In this Example we have evaluated the safety of a pigmented strain of Bacillus, HU36, as an illustration of the safety of the Bacillus strains of the invention. HU36 produces spores that are rich in carotenoids and could provide a natural source or carotenoids to the human or animal diet. The results obtained show the potential for use of these bacteria as food supplements.

### Materials and Methods

### Bacterial Strains

HU36 is a yellow-orange pigmented spore-forming species of Bacillus. Reference strains used in this work were PY79 a prototrophic strain of Bacillus subtilis derived from the 168 type-strain (Youngman et al., 1984); SC2329, a toxin producing strain of B. cereus (Hoa et al., 2000) and, Natto, a laboratory strain (SC2404) of B. subtilis var Natto obtained from a sample of the Japanese staple Natto.

### General Methods and Preparation of Spores

Purified suspensions of spores were made by the exhaustion method (Nicholson and Setlow, 1990) using DSM (Difco Sporulation) medium (Duc et al., 2003). Spore suspensions were lysozyme-treated and then heat-treated (68° C. 1 h) to remove residual vegetative cells and stored as aliquots at −20° C. prior to use.

### Analysis of Enterotoxins and Virulence Traits

Methods to detect putative B. cereus enterotoxin genes from Bacillus species by PCR amplification from chromosomal DNA have been described in detail elsewhere (Duc et al., 2004). Primer sets were those described by Guinebretiere et al. (Guinebretiere et al., 2002). The Hbl and Nhe enterotoxins were detected and measured using commercial kits; the BCET-RPLA kit (Oxoid) to detect Hbl and the Tecra BDE kit (Tecra Diagnostics) to detect the Nhe enterotoxin. Hemolysis was detected by streaking on sheep blood agar plates and lecithinase by streaking colonies on B. cereus selective agar containing egg yolk and 48 h incubation at 37° C.

### Adhesion and Invasion Studies

Adhesion of spores or vegetative cells was determined using three different cell-lines, Caco-2 (human colon cancer cells), HEp-2 and mucin-producing HT29 (human Caucasian colon adenocarcinoma) cells obtained from the European Collection of Cell Cultures (ECCAC), UK. In each case cells were sown at 2×10⁵ cells per well in 24-well chamber slides (Nunc™) in MEME (Minimal Essential Medium Eagle; Sigma) supplemented with foetal calf serum 10% (v/v), L-glutamine 1% (v/v) and non-essential amino acids 1% (v/v) for 2 days at 37° C. in 5% CO₂. The adhesion and invasion assays were as described in detail in Rowan et al. (Rowan et al., 2001).

### Cytotoxicity Assay

The assay was as described by Rowan et al (Rowan et al., 2001) using Hep-2 or Caco-2 cell monolayers seeded at 5×10⁴ cells per well. Cells were infected with filter-sterilised (0.2 mm) supernatants from overnight cultures of bacteria to be tested. Samples of supernatant (0.1 ml) were added immediately in triplicate to cultured cells, after trypsin-treatment (0.1% trypsin; 5 min) or after heat-treatment (95° C., 10 min.). Monolayers containing the bacterial supernatants were incubated overnight at 37° C. in a 5% CO₂ atmosphere. After overnight incubation phosphate buffered saline containing 0.5% MTT (3-(5-dimethylthiazole-2-yl)-2,5-diphenyl tetrazolium bromide; Sigma) and incubation continued for 4 h. The suspensions from each well were then removed, and the formazan produced contained in each well solubilised by the addition of 100 ml of 0.04M HCl in dimethyl sulfoxide and measured spectrophotometrically at 540 nm with a microplate reader. Toxicity was measured as follows; (1-optical density of test sample/optical density of negative control)×100.

### Anaerobic Growth

The procedure to evaluate the ability of Bacillus strains to grow and sporulate under anaerobic conditions has been described elsewhere (Tam et al., 2006).

### Persistence Studies

Mice were housed in cages with gridded floors to prevent coprophagia. A single dose (0.2 ml) of 1×10⁹ spores was given to mice by oral gavage. For sampling, individual mice were removed and held until a single fresh fecal pellet was collected, weighed by difference and stored at −20° C. before analysis of heat-resistant cfu/g as described (Duc et al., 2004).

### Simulated Intestinal Conditions

Spores and vegetative cells were evaluated using the method described in Barbosa et al (Barbosa et al., 2005). For simulated bile fluid (SIF) spores or vegetative cells were resuspended (at a density of 1×10⁸ cfu/ml) in an isotonic buffer (Bott and Wilson salts) containing 0.2% bile salts or for simulated gastric fluid (SGF) in 0.85% NaCl (pH 2) containing 1 mg/ml pepsin, Suspensions were incubated at 37° C. for 2 h and sampled periodically for cfu/ml by serial dilution and plate counting on LB agar plates.

### Biofilm Formation

Biofilm formation was determined using the method of Fall (Fall et al., 2004) using growth on CMK agarose or in CMK liquid medium and growth at 37° C. for 2-3 days in each case.

### Antibiotic Testing

Antibiograms for strains were obtained by the disc diffusion method according to the recommendations of the National Committee for Clinical Laboratory Standards (NCCLS; (Standards, 1997)). Overnight broth cultures of tested strains after growth in LB broth at 37° C. were seeded on Mueller-Hinton plates by swab inoculation. Antibiotic-impregnated discs (Oxoid; 6 mm) were placed on seeded plates and the zone of inhibition was measured after 18 h at 37° C.

### Subchronic Toxicity Test

This test (also known as the continuous exposure test) was carried out at the Institute of Vaccines and Biological Substances (IVAC) in Nha Trang, Vietnam. The test was made using New Zealand White rabbits (male, 3 months old) divided into three groups of six animals. Two groups were orally dosed with 1 ml of a purified suspension of spores (1×10⁹/ml). One group received HU36 spores and the other Natto. The naïve group received 1 ml of saline. Animals were dosed using this regime daily for 30 days. On day 31 blood was withdrawn for hematological analysis and animals were then euthanised humanely and samples of different organs and tissues collected for histological analysis including liver, kidneys, spleens, small intestines and mesenteric lymph nodes.

### Acute Toxicity Study

Animals used in this test were guinea-pigs (Harley Dunkin, male, 5 weeks old) and the test carried out at IVAC, Nha Trang, Vietnam. A single 1 ml dose of 1×10¹² spores of either HU36 or Natto was administered orally to groups of 6 animals. A naïve group received 1 ml of saline. Animals were observed daily for seven days and behaviour, appearance, activity and faeces recorded. Body weight was recorded on day 1 and day 7. On day 8 blood was withdrawn for hematological analysis and animals were then euthanised humanely and samples of different organs and tissues collected for histological analysis including liver, kidneys, spleens, small intestines and mesenteric lymph nodes.

### Histology

Samples of organs and tissues were fixed in 10% formalin, transferred into ethanol solutions with increasing concentrations and embedded in paraffin wax. Tissue sections were cut at 6 mm and stained with haematoxilin and eosin.

### Haematology

In rabbits blood samples were obtained from rabbits and guinea pigs by cardiac puncture and total red blood cells (RBC), leukocytes, haemoglobin concentration and differential percentages of white blood cells determined.

### Plasmids Analysis

Plasmid DNA was extracted from the Bacillus strains using the method of Voskuil and Chambliss (1993) method. The samples were run at 10 V/cm in 1.0% agarose gels (1×TBE buffer: 89 mM Tris, 89 mM boric acid, 2 mM EDTA) containing (0.25 mg/ml) ethidium bromide and photographed under UV light.

### Statistics

The data were analysed using Student's t-test and the Fisher exact test. P values less than 0.05 were considered significant.

### Results

To evaluate HU36 as an illustratory examples of Bacilli of the invention, the carotenoid-rich, pigmented Bacillus species evaluations made on HU36 were run in parallel to two isolates of B. subtilis; PY79, a laboratory strain derived from the 168 type-strain (Youngman et al., 1984), and Natto, a strain of B. subtilis used in the Japanese fermented food known as ‘Natto’ (Hosoi and Kiuchi, 2004).

### Sporulation Properties

The ability of HU36 to grow and sporulate was examined in detail (Table 6). In a previous study it was found that HU36 could not grow anaerobically when streaked on nutrient rich agar. However, when grown in liquid culture and then plated onto DSM agar plates HU36 was found to be able to grow anerobically as has been shown previously for strains of B. subtilis (Tam et al., 2006). Growth was ten-fold higher when nitrite or nitrate as the terminal electron acceptor was present. Under anoxic conditions though, HU36 cells could not form spores in any significant level as was also the case with PY79. Natto, though, could sporulate relatively efficiently under these conditions and at higher levels when in the presence of nitrite or nitrate.

The resistance of spores of HU36, Natto and PY79 to exposure to simulated intestinal fluids was also measured (Table 7). Neither PY79 nor Natto showed any measurable sensitivity to simulated gastric fluid (SGF) or simulated intestinal fluid (SIF). HU36, though, did show some sensitivity to both SGF and SIF but percentage survival was still high.

In Table 6 Bacillus strains were grown in liquid DSM and plated on DSM agar plates. For anaerobic growth, potassium nitrate or potassium nitrite was added to the medium as an electron acceptor. Plates were incubated for 72 h at 30° C. anaerobically or aerobically after which the entire bacterial lawn was recovered from each plate. The suspension was then serially diluted and plated out for CFU or heat-treated (68° C., 45 min.) before serial dilution to determine spore counts.

TABLE 6
Sporulation efficiency under aerobic and anerobic conditions^{a}.

| | | | | | Anaerobic | | |
|---|---|---|---|---|---|---|---|
| | Growth | Aerobic | | | | Spore | |
| | conditions | Total count | Spore count | Sporulation | Total count | count | Sporulation |
| Strain | Parameter | (CFU/plate) | (CFU/plate) | efficiency (%) | (CFU/plate) | (CFU/plate) | efficiency (%) |
| PY79 | DSM | 1.81 × 10¹⁰ | 1.56 × 10¹⁰ | 86.19 | 2.05 × 10⁷ | 23 | 1.12 × 10⁻⁴ |
| | DSM + | 2.09 × 10¹⁰ | 1.92 × 10¹⁰ | 91.87 | 2.92 × 10⁷ | 141 | 4.83 × 10⁻⁴ |
| | NO₂ | | | | | | |
| | DSM + | 1.72 × 10¹⁰ | 1.63 × 10¹⁰ | 94.77 | 2.68 × 10⁷ | 216 | 8.06 × 10⁻⁴ |
| | NO₃ | | | | | | |
| HU36 | DSM | 1.74 × 10¹⁰ | 1.19 × 10¹⁰ | 68.39 | 8 × 10⁵ | 12 | 1.5 × 10⁻³ |
| | DSM + | 1.85 × 10¹⁰ | 1.24 × 10¹⁰ | 67.03 | 1.24 × 10⁶ | 47 | 3.79 × 10⁻³ |
| | NO₂ | | | | | | |
| | DSM + | 1.56 × 10¹⁰ | 1.01 × 10¹⁰ | 64.74 | 1.53 × 10⁶ | 52 | 3.39 × 10⁻³ |
| | NO₃ | | | | | | |
| Natto | DSM | 2.12 × 10¹⁰ | 2.11 × 10¹⁰ | 99.53 | 1.55 × 10⁷ | 4.28 × 10⁴ | 0.28 |
| | DSM + | 2.41 × 10¹⁰ | 2.37 × 10¹⁰ | 98.34 | 2.93 × 10⁷ | 1.23 × 10⁶ | 4.20 |
| | NO₂ | | | | | | |
| | DSM + | 2.06 × 10¹⁰ | 2.03 × 10¹⁰ | 98.54 | 3.03 × 10⁷ | 1.71 × 10⁶ | 5.64 |
| | NO₃ | | | | | | |

TABLE 7
Resistance of vegetative and cells in simulated GIT fluids

| | | % survival^{a} | | | |
|---|---|---|---|---|---|
| Bacillus strain^{b} | Treatment | 0 min. | 15 min. | 30 min. | 60 min. |

Simulated gastric fluid

| | | | | | |
|---|---|---|---|---|---|
| PY79 | none | 100 | 102 | 105 | 106 |
| (4.22 × 10⁸) | +SGF | 100 | 99 | 96 | 94 |
| Natto | none | 100 | 104 | 102 | 101 |
| (5.85 × 10⁸) | +SGF | 100 | 98 | 96 | 97 |
| HU36 | None | 100 | 99 | 97 | 93 |
| (4.12 × 10⁸) | +SGF | 100 | 85 | 82 | 80 |

Simulated intestinal fluids

| | | | | | |
|---|---|---|---|---|---|
| PY79 | none | 100 | 106 | 107 | 108 |
| (4.22 × 10⁸) | +SIF | 100 | 101 | 98 | 96 |
| Natto | none | 100 | 101 | 105 | 105 |
| (5.85 × 10⁸) | +SIF | 100 | 98 | 99 | 101 |
| HU36 | None | 100 | 93 | 88 | 86 |
| (4.12 × 10⁸) | +SIF | 100 | 80 | 75 | 80 |

^{a}percentage of cfu surviving relative to initial cfu. Surviving at the indicating time (minutes).
^{b}The cfu of bacterial suspension of spores or vegetative cells is shown in brackets

TABLE 8
Potential virulence characteristics

Bacillus strains

| | | | | |
|---|---|---|---|---|
| Characteristic | HU36 | PY79 | Natto | SC2329 |

| Hemolysis^{a} | | γ | γ | γ | β |
|---|---|---|---|---|---|
| Lecithinase^{b} | | − | − | ± | + |
| Biofilm formation | | − | − | − | − |
| Hbl Complex^{c} | hblA | − | − | − | + |
| | hblB | − | − | − | − |
| | hblC | − | − | − | + |
| | hblD | − | − | − | + |
| HBL Enterotoxin^{d} | | 0 | 0 | 0 | 128 |
| Nhe complex^{c} | nheA | − | − | − | + |
| | nheB | − | − | − | + |
| | nheC | − | − | − | + |
| NHE Enterotoxin^{d} | | 1 | 1 | 1 | 4 |
| Other | cytK | − | − | − | + |
| enterotoxin | bceT | − | − | − | + |
| genes^{d} | | | | | |

^{a}hemolysis on sheep blood agar; β, complete hemolysis with a clear zone around colonies; γ no changes.
^{b}lecithinase production; + blue precipitation of hydrolysed lecithin around peacock blue colonies (indicative of B. cereus); −, no changes; ±, weak coloration.
^{c}Genes encoding components of the Hbl or Nhe enterotoxins or other B. cereus enterotoxins were diagnosed by PCR; +, a PCR product of the expected size was amplified; −, no PCR product was detected.
^{d}Production of the Hbl enterotoxin in growing cells was determined using the BCET-RPLA toxin kit (Oxoid) and expressed as an index where a value of 0 is negative according to the manufacturer's instructions. The sensitivity of the test is 2 ng/ml.
^{e}Production of the Nhe enterotoxin in growing cells was measured using the ‘Bacillus Diarrhoeal Enterotoxin Visual Immunoassay kit’ (Tecra Diagnostics). According to the manufacturer's instructions, strains with an index of <3 are considered negative and the sensitivity of the test is 1 ng/ml.

### Antibiotic Resistance

HU36 was evaluated for its resistance to a panel of antibiotics including those highlighted by the Scientific Committee on Animal Nutrition (SCAN) and the European Food Standard Agency (EFSA) (EFSA, 2005; SCAN, 2003b). Antimicrobial resistance was determined in two ways, first using agar disc-diffusion assay (Table 9) and second by establishing the MIC (Table 10). HU36 was found to carry noticeable resistance only to clindamycin which was above the published MIC breakpoint for this compound (EFSA, 2005). In an attempt to determine whether this resistance might have been acquired we attempted to isolate plasmid DNA from HU36, PY79 and Natto and failed to isolate any DNA that might correspond to episomal DNA.

TABLE 9
Antibiotic resistance profiles of Bacillus strains.

| | Zone of inhibition^{b} (mm) | | |
|---|---|---|---|
| Antibiotic^{a} | PY79 | Natto | HU36 |
| Ampicillin (10 μg) | 29.5 ± 0.5 | 31.7 ± 1.5 | 31.7 ± 1.5 |
| Chloramphenicol (30 μg) | 25.3 ± 1.3 | 26.7 ± 1.2 | 24.3 ± 1.3 |
| Ceftazidime (30 μg) | 26.7 ± 0.6 | 23.3 ± 1.5 | 20.8 ± 1 |
| Cefaclor (30 μg) | 40.7 ± 1.5 | 36.3 ± 2.5 | 31.7 ± 0.6 |
| Ciporfloxacin (5 μg) | 32.8 ± 1 | 33.3 ± 1.5 | 29.7 ± 0.6 |
| Ceftriaxone (30 μg) | 30.1 ± 0.7 | 27.7 ± 1.5 | 24.7 ± 1.3 |
| Clindamycin (2 μg) | 22.3 ± 1.2 | 22.7 ± 1.5 | 10 ± 0.9 |
| Erythromycin (15 μg) | 25.3 ± 1.2 | 25.3 ± 1.2 | 25.7 ± 1.2 |
| Imipenem (10 μg) | 43.7 ± 2.5 | 39 ± 1 | 30.7 ± 1.8 |
| Kanamycin (30 μg) | 28.3 ± 1.3 | 24.3 ± 1.5 | 22.1 ± 0.7 |
| Naladixic acid (30 μg) | 20.7 ± 1.2 | 23 ± 1.7 | 22.8 ± 1.4 |
| Rifampicin (5 μg) | 21.7 ± 0.6 | 31.3 ± 1.5 | 21.7 ± 1.2 |
| Tetracycline (30 μg) | 19.7 ± 1.3 | 28.7 ± 1.5 | 31.2 ± 0.8 |
| Vancomycin (30 μg) | 20.3 ± 1.5 | 21.7 ± 1.5 | 20.3 ± 0.6 |
| Trimethoprim (23.75 | 28.8 ± 1 | 33.3 ± 0.6 | 28.2 ± 0.8 |
| μg)/Sulfamethoxazole | | | |
| (1.25 μg) | | | |

^{a}antibiotic-impregnated discs (6 mm) with amount, in μg shown in brackets.
^{b}zones of inhibition (diameter) from three individual experiments.

TABLE 10
MICs

| | |
|---|---|
| MICs (mg/l) | Breakpoints^{a} |

| Antibiotic | PY79 | HU36 | SCAN^{b} | EFSA^{c} |
|---|---|---|---|---|
| Ampicillin | | | 2^{d} | NR^{e} |
| Chloramphenicol | | | 16 | 8 |
| Ceftazidime | 0.025 | 0.05 | | |
| Cefaclor | | | | |
| Ciporfloxacin | | | | |
| Ceftriaxone | | | | |
| Clindamycin | 0.05 | 8 | — | 4 |
| Erythromycin | | | 4 | 4 |
| Gentamycin | | | 8 | 4 |
| Imipenem | | | | |
| Kanamycin | | | 64 | 8 |
| Naladixic acid | | | | |
| Neomycin | | | — | 8 |
| Rifampicin | | | 324 | — |
| Streptomycin | | | 64 | 8 |
| Tetracycline | | | 16 | 8 |
| Vancomycin | | | 4 | 4 |
| Trimethoprim/ | | | | |
| Sulfamethoxazole | | | | |

^{a}Strains with MICs equal or higher are considered resistant.
^{b}(SCAN, 2002)
^{c}(EFSA, 2005).
^{d}Certain species are inherently resistant.
^{e}Breakpoint not required.

### Adhesion Assays

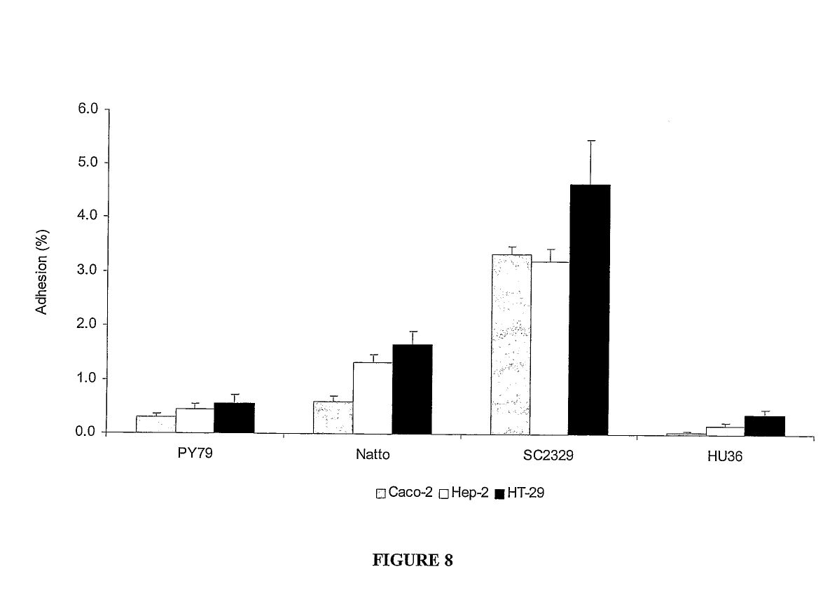

Adhesion of HU36, PY79, Natto and SC2329 to adhere to Caco-2, Hep-2 and HT29 cells was evaluated using in vitro methods. HT29 cells exhibit differentiation features characteristic of mature intestinal cells and are therefore more informative than Caco-2 cells (Devine et al., 1992). In each case a suspension of 1×10⁸ spores of each strain was incubated for two hours with the cultured cell line under conditions in which germinated spores or live vegetative bacteria would be killed. The results obtained are shown in FIG. 8. SC2329 exhibited the highest levels of adhesion followed by Natto and PY79 and finally HU36 which exhibited very low levels of adhesion. In each case though adhesion was always greatest to the mucin-producing HT29 cell-line.

### Persistence of Spores in the Mouse GIT

The shedding of HU36, PY79 and SC2329 spores in the faeces of mice given a single oral dose of spores was measured. The results obtained show counts of PY79 and HU36 spores were no longer within detectable levels after 12 days while SC2329 persisted for three weeks.

### Potential Virulence Factors

PCR was used to evaluate the presence of known B. cereus enterotoxin genes in the chromosome of HU36, PY79, Natto and as a control SC2329. This method has been used previously to profile putative food-poisoning Bacillus strains (Duc et al., 2004; Guinebretiere et al., 2002; Phelps and McKillip, 2002). With the exception of SC2329 that was a B. cereus control, none of the known B. cereus enterotoxin genes were detected. In vivo analysis was also made for the Hbl and Nhe B. cereus enterotoxins also tested negative. Haemolysis was also not produced on sheep's blood agar by HU36, PY79 and Natto. Production of lecithinase (a phospholipase) was also negative in HU36 and PY79 although a weak reaction was observed with Natto.

### Cytoxicity Assays

The toxicity of supernatants of growing cultures of HU36, PY79, Natto and SC2329 was evaluated using a spectrophotometric assay to measure cell death following exposure of either Caco-2 or Hep-2 cells. Our results shown in Table 11 demonstrate that HU36 supernatants contained some toxigenic potential which was both heat and trypsin stable. This was markedly less than the B. cereus control strain, SC2329. By contrast though, PY79, the lab strain demonstrated little to no toxigenic potential while Natto carried some trypsin-sensitive toxic material in it supernatant.

TABLE 11
Effect of heat or trypsin treatment on cytotoxicity
of cell-free supernatants on Hep-2 epithelial cells.

| | Hep-2 % cell death | | |
|---|---|---|---|
| Bacillus pp. | Normal | HT | TT |
| PY79 | 8 | 3 | 7 |
| Natto | 43 | 31 | 10 |
| SC2329 | 97 | 84 | 86 |
| HU36 | 73 | 63 | 45 |

^{a}supernatant fluids were untreated (Normal), heat-treated (HT) or trypsin-treated (TT).

### In Vivo Toxicity Assays

Two assessments of the safety of HU36 in animals was made. In the first test, the sub-chronic toxicity test, rabbits were administered, orally, with 1×10⁹ spores of HU36 or Natto. The same daily dosing was then maintained for 30 days. In the second test, the acute toxicity test, a single large dose of 1×10¹² spores was administered to guinea-pigs by the oral route and then observed for 7 days.

In either test, no sign of illness or behavioural changes were observed. In the acute toxicity test there were body weight gains (Table 12) with animals receiving HU36 experiencing an average weight gain of 60% and those receiving Natto, 20%. With the sub chronic toxicity test though rabbits showed no change in their body weights compared to the untreated animals. The weights of livers, spleens and kidneys were similar to animals receiving no spores (Table 12). No sign of pathology could be found in the organs and tissues of animals including the absence of inflammation, degeneration or necrosis (data not shown). Haematological analysis of blood from both tests were made in full (Table 13) and no differences between groups could be found.

TABLE 12
Body weights from in vivo testing.
Sub-chronic toxicity test

| | Mean of body weight (kg) | | | | |
|---|---|---|---|---|---|
| Groups^{a} | Day 0 | Day 8 | Day 16 | Day 24 | Day 31 |
| Control | 2.15 ± 0.13 | 2.23 ± 0.10 | 2.43 ± 0.06 | 2.15 ± 0.13 | 2.52 ± 0.11 |
| Natto | 2.25 ± 0.2 | 2.39 ± 0.15 | 2.55 ± 0.16 | 2.15 ± 0.13 | 2.67 ± 0.11 |
| HU36 | 2.19 ± 0.12 | 2.34 ± 0.10 | 2.55 ± 0.05 | 2.15 ± 0.13 | 2.62 ± 0.08 |

| | Average weight of organs (g) | | |
|---|---|---|---|
| | Liver | Spleen | Kidneys |
| Control | 55.43 ± 5.2 | 1.31 ± 0.13 | 5.39 ± 0.25 |
| Natto | 55.16 ± 7.0 | 1.39 ± 0.15 | 5.33 ± 0.2 |
| HU36 | 55.25 ± 7.9 | 1.37 ± 0.13 | 5.33 ± 0.29 |

Acute toxicity test

Average weights

| Groups^{b} | Day 0 | Day 7 | AWG^{c} | AWG %^{d} |
|---|---|---|---|---|
| Control | 288 | 308 | 20 | 100 |
| Natto | 297 | 321 | 24 | 120 |
| HU36 | 300 | 332 | 32 | 160 |

Average weight of organs (g)

| | Liver | Spleen |
|---|---|---|
| Control | 13.23 ± 0.32 | 0.496 ± 0.039 |
| Natto | 13.41 ± 0.55 | 0.496 ± 0.044 |
| HU36 | 13.65 ± 0.58 | 0.479 ± 0.074 |

^{a}groups of six rabbits dosed orally, every day, for 30 days with 1 × 10⁹ spores of PY79, Natto or HU36, or a naïve group (Control).
^{b}groups of six guinea pigs administered with a single oral dose of 1 × 10¹² spores.
^{c}average weight gain.
^{d}average weight gain compared to the control group.

TABLE 13
Hematology analysis.

| | Sub-chronic test | | | Acute toxicity test | | |
|---|---|---|---|---|---|---|
| Parameter | Control^{a} | Natto | HU36 | Control^{a} | Natto | HU36 |
| Red blood cells ×10¹² | 5.26 ± 0.37 | 4.73 ± 0.69 | 5.8 ± 0.21 | 4.76 ± 0.397 | 4.86 ± 0.37 | 4.78 ± 0.35 |
| Hemoglobin(g/l) | 118 ± 14.20 | 131.05 ± 13.7 | 109.44 ± 8.50 | 128.42 ± 7.50 | 131.92 ± 4.79 | 132.90 ± 5.52 |
| Hematocrite (l/l) | 0.343 ± 0.03 | 0.39 ± 0.02 | 0.37 ± 0.02 | 0.369 ± 0.027 | 0.38 ± 0.024 | 0.39 ± 0.018 |
| Platelets ×10⁹ | 383 ± 110.7 | 370.35 ± 93.52 | 594.81 ± 30.23 | 391.56 ± 66.02 | 379.80 ± 91.9 | 334.94 ± 56.25 |
| White blood | 4.49 ± 1.61 | 4.38 ± 1.73 | 5.95 ± 0.92 | 3.59 ± 0.57 | 4.09 ± 0.50 | 3.30 ± 0.28 |
| cells ×10⁹ | | | | | | |
| Neutrophils (%) | 63.8 ± 3.25 | 56.11 ± 3.00 | 60.21 ± 3.27 | 53.00 ± 5.60 | 52.86 ± 5.80 | 54.05 ± 2.45 |
| Lymphocytes (%) | 32.15 ± 1.63 | 36.6 ± 1.80 | 34.31 ± 2.26 | 40.88 ± 2.77 | 39.77 ± 4.71 | 36.63 ± 5.69 |
| Monocytes (%) | 2.05 ± 1.72 | 3.65 ± 1.52 | 2.84 ± 0.86 | 5.28 ± 4.35 | 6.36 ± 4.53 | 5.02 ± 3.42 |
| Eosinophils (%) | 0.87 ± 0.06 | 1.54 ± 1.13 | 1.76 ± 0.05 | 1.36 ± 0.94 | 0.82 ± 0.59 | 3.60 ± 0.63 |
| Basophils (%) | 1.23 ± 0.42 | 1.85 ± 0.32 | 2.40 ± 0.02 | 0.3 ± 0.08 | 0.54 ± 0.16 | 1.22 ± 0.28 |

^{a}a naïve group receiving no spores

### Discussion

The initial part of this work was to evaluate what might happen to ingested spores of HU36. Studies on B. subtilis spores have shown that they germinate in the GIT of mice, replicate and sporulate (Casula and Cutting, 2002; Hoa et al., 2001; Tam et al., 2006). Indeed, the studies show that spores are seemingly well designed for transit through the gut. Growth of Bacillus species under anaerobic conditions is a characteristic reported only recently and a phenomenon that does not occur with all species (Nakano et al., 1997; Nakano and Zuber, 1998). The B. subtilis strain PY79 that is derived from the 168-type strain and used in many laboratories can grow anaerobically but can not sporulate under anoxic conditions (Tam et al., 2006). However, in the same study certain human isolates of B. subtilis (HU58 and HU78) were found able to sporulate efficiently under anoxic conditions. Despite this sporulation of PY79 does occur inside the GIT of the mouse since mice dosed with PY79 spores shed more in their faeces (Hoa et al., 2001) than administered which suggests that the GIT is not entirely anaerobic. In this study HU36 can grow efficiently under anaerobic conditions but, like PY79, cannot sporulate. A second control strain, B. subtilis Natto could sporulate at much higher levels.

Spores of HU36 were essentially unaffected by exposure to simulated gastric and bile fluids. Not all Bacillus spores are resistant to gastric fluids, most notably, some strains of B. cereus are acutely sensitive to stomach acidity and in this case stimulate germination of the spore and release of the vegetative cell and a premature demise in the stomach acids (Faille et al., 2002; Keynan and Evenchik, 1969).

Many strains of B. cereus are known to carry genes that encode one or more of four known enterotoxins (Hbl, Nhe, CytK and BceT) but it is plausible that one or more of these genes may be present on the genomes of other Bacillus species (Guinebretiere et al., 2002; Rowan et al., 2001). Establishing that strains to be used as food supplements are incapable of producing toxins is obviously of importance and both B. subtilis strains as well as HU36 were devoid of known toxin genes. Other potential virulence factors were also observed including haemolysis, the ability to form biofilms and the production of the phospholipase lecithinase. The latter of which did give a weak reaction with the control food strain of B. subtilis, Natto.

HU36 was found to carry resistance to only one antibiotic, clindamycin. The MIC was at a level than the EU-recommended breakpoint for this compound. However, no plasmid could be recovered from HTU36, Natto nor PY79 and it seems improbable that this resistance-determinant is acquired and therefore satisfies one of the published requirements for QPS for microbial food supplements (EFSA, 2005).

Assays for the cytotoxic effects of cell-free supernatants revealed that while the laboratory strain PY79 had essentially no toxic effect on cultured cells and the B. cereus strain SC2329 was noticeably toxic, strain HU36 had some intermediate effect on cell monolayers. This effect appeared partially trypsin-sensitive. In other work it has been shown that cytotoxic effects on cultured cells is a problematic determinant and may not correlate with the production of enterotoxins (From et al., 2005; Rowan et al., 2001). Indeed, it is possible that toxic effects observed in vitro may result from the production of excessive amounts of proteases or even antimicrobials, such as the lipopeptide, Surfactin (Nagal et al., 1996). This has lead the EU to conclude that the measurement of cytoxicity may be impaired by the effects of low molecular weight material in culture supernatants (SCAN, 2001). Thus, such low levels of toxicity in vitro are not problematic.

To determine whether oral consumption of HU36 spores produced any toxicity we performed two safety tests using animals. The sub-chronic toxicity test measured the effect of daily oral doses (1×10⁹) of HU36 and Natto spores in rabbits for 30 days and no ill-effect was observed. The acute toxicity test measured the effect of a single oral dose of 1×10¹² spores of HU36 and Natto in guinea pigs. As with the sub-chronic toxicity test no deleterious effect on animals with no impairment of general health, behaviour or histological changes in the organs and tissues. The acute toxicity test then demonstrated that in guinea pigs the oral LD₅₀ must be greater than 1×10¹² for both Natto and HU36.

In vitro adhesion assays determined that spores of HU36, had limited ability to bind to any of the epithelial cell lines tested. Binding of PY79 was somewhat higher and with Natto better still. This was in agreement with studies made on the adhesion of Bacillus species to Caco-2 and Hep-2 cells with the conclusion that B. subtilis adheres poorly to epithelial cell lines (Rowan et al., 2001). B. cereus though which was used as a control in these experiments bound even better with almost ten-times greater binding. In all cases though binding was best to the mucin-producing HT29 cell line.

Experiments in vivo also examined the fate of spores administered as a single dose to mice. These persistence experiments showed that HU36 was shed from the GIT after 14 days and no longer present within the animal at detectable levels. This correlated well with similar studies examining the persistence of the laboratory strain, PY79, of B. subtilis and confirmed here. On the other hand, the B. cereus strains used here persisted for a further seven days within the gut. A similar phenomenon has also been observed for two human isolates of B. subtilis, HU58 and HU78 (Tam et al., 2006) and suggests that natural B. subtilis isolates or species such as B. cereus that are known to live within the GIT are better adapted to the gut environment.

The outcome of these assays is that HU36 is not particularly well adapted to the gut environment. It binds to epithelial cells poorly and does not sporulate under anoxic conditions. In these regards it is similar to the laboratory strain of B. subtilis, PY79. On the other hand shows no virulent characteristics nor is it toxigenic in animal models. As a potential food supplement then it shows no sign of being pathogenic or harmful to man.

The origin of HU36 was from human faeces obtained from volunteers in Vietnam. In reality this bacterium may have originated from the diet that is rich in seafood since pigmented bacilli are common in shellfish, most notably shrimps. If correct, this halotolerant spore-former, may not be suited for survival in the human GIT.

The experiments performed therefore show that:
1. in vitro studies demonstrate that HU36 as an illustratory strain of the invention shows no toxicity in rabbits or guinea-pigs when used orally. The oral LD₅₀ must be greater than 1×10¹² cfu.
2. There are no virulence characteristics, including toxin genes, toxin production, haemolysis etc.
3. Resistance to only one antibiotic was found, clindamycin and there is no evidence this was acquired and this is not plasmid borne.
4. HU36 adheres poorly to epithelial cells and does not persist long in the GIT.
   In summary, for the Bacilli of the invention, such as, HU36, there is no evidence that they are toxic or pathogenic. The Bacilli of the invention may therefore be considered harmless and suitable for human use.

### REFERENCES

Ash, C. J., Farrow, J. A. E., Wallbanks, S., and Collins, M. D., (1991) Phylogenetic heterogeneity of the genus Bacillus revealed by comparative analysis of small-subunit-ribosomal RNA sequences. Letters in Applied Microbiology. 13: 202-206.
Badenhop, F., Steiger, S., Sandmann, M., and Sandmann, G. (2003) Expression and biochemical characterization of the 1-HO-carotenoid methylase CrtF from Rhodobacter capsulatus. FEMS Microbiol Lett 222: 237-242.
Britton, G., Liaaen-Jenson, S., and Pfander, H. (eds) (2003) Carotenoids, Handbook. Basel, Switzerland: Birkhauser Verlag.
Fraser, P. D., Pinto, M. E., Holloway, D. E., and Bramley, P. M. (2000) Technical advance: application of high-performance liquid chromatography with photodiode array detection to the metabolic profiling of plant isoprenoids. Plant J 24: 551-558.
Hoa, N. T., Baccigalupi, L., Huxham, A., Smertenko, A., Van, P. H., Ammendola, S. et al. (2000) Characterization of Bacillus species used for oral bacteriotherapy and bacterioprophylaxis of gastrointestinal disorders. Appl. Env. Microbiol. 66: 5241-5247.
Martin, V. J., et al (2003) Engineering a mevalonate pathway in Eschericia coli for production of terpenoids. Nature Biotechnology, 21(7): 796-802.
Nicholson, W. L., and Setlow, P. (1990) Sporulation, germination and outgrowth. In Molecular Biological Methods for Bacillus. Harwood., C. R., and Cutting., S. M. (eds). Chichester, UK.: John Wiley & Sons Ltd., pp. 391-450.
Rotman., B., and Cote, M. A., (2003) Application of real time biosensor to detect bacteria in platelet concentrates. B.B.R.C., 300: 197-200.
Suresh, K., Prabagaran, S. R., Sengupta, S., and Shivaji, S. (2004) Bacillus indicus sp. nov., an arsenic-resistant bacterium isolated from an aquifer in West Bengal, India. Int J Syst Evol Microbiol 54: 1369-1375.
Youngman, P., Perkins, J., and Losick, R. (1984) Construction of a cloning site near one end of Tn917 into which foreign DNA may be inserted without affecting transposition in Bacillus subtilis or expression of the transposon-borne erm gene. Plasmid 12: 1-9.

### FURTHER REFERENCES

anon. (2004) Opinion of the Scientific Panel on Biological Hazards on Bacillus cereus and other Bacillus spp in foodstuffs. The EFSA Journal 175: 1-48.
Barbosa, T. M., Serra, C. R., La Ragione, R. M., Woodward, M. J., and Henriques, A. O. (2005) Screening for bacillus isolates in the broiler gastrointestinal tract. Appl Environ Microbiol 71: 968-978.
Borrielo, S. P., Hammes, W. P., Holzapfel, W., Marteau, P., Schrezenmeir, J., Vaara, M., and Valtonen, V. (2003) Safety of probiotics that contain Lactobacilli of Bifidobacteria. Clinical Infectious Diseases 36: 775-780.
Casula, G., and Cutting, S. M. (2002) Bacillus probiotics: spore germination in the gastrointestinal tract. App. Env. Microbiol. 68: 2344-2352.
de Boer, A. S., and Diderichsen, B. (1991) On the safety of Bacillus subtilis and B. amyloliquefaciens: a review. Applied Microbiology and Biotechnology 36: 1-4.
Devine, P. L., Birrell, G. W., Whitehead, R. H., Harada, H., Xing, P. X., and McKenzie, I. F. (1992) Expression of MUC1 and MUC2 mucins by human tumor cell lines. Tumour Biol 13: 268-277.
Duc, L. H., Hong, H. A., Fairweather, N., Ricca, E., and Cutting, S. M. (2003) Bacterial spores as vaccine vehicles. Infection and Immunity 71: 2810-2818.
Duc, L. H., Hong, H. A., Barbosa, T. M., Henriques, A. O., and Cutting, S. M. (2004) Characterization of Bacillus Probiotics Available for Human Use. App. Environ. Microbiol. 70: 2161-2171.
Duc, L. H., Dong, T. C., Logan, N. A., Sutherland, A. D., Taylor, J., and Cutting, S. M. (2005) Cases of emesis associated with bacterial contamination of an infant breakfast cereal product. International Journal of Food Microbiology 102: 245-251.
EFSA (2005) Opinion of the Scientific Committee on a request from EFSA related to a generic approach to the safety assessment by EFSA of microorganisms used in food/feed and the production of food/feed additives. The EFSA Journal 226: 1-12.
Faille, C., Membre, J., Kubaczka, M., and Gavini, F. (2002) Altered ability of Bacillus cereus spores to grow under unfavorable conditions (presence of nisin, low temperature, acidic pH, presence of NaCl) following heat treatment during sporulation. Journal of Food Protection 65: 1930-1936.
Fall, R., Kinsinger, R. F., and Wheeler, K. A. (2004) A simple method to isolate biofilm-forming Bacillus subtilis and related species from plant roots. Syst Appl Microbiol 27: 372-379.
FAO/WHO (2002) Joint FAO/WHO (Food and Agriculture Organization/World Health Organization) working group report on drafting guidelines for the evaluation of probiotics in food. London, Ontario, Canada.
From, C., Pukall, R., Schumann, P., Hormazabal, V., and Granum, P. E. (2005) Toxin-producing ability among Bacillus spp. outside the Bacillus cereus group. Appl Environ Microbiol 71: 1178-1183.
Guinebretiere, M.-H., Broussolle, V., and Nguyen-The, C. (2002) Enterotoxigenic profiles of food-poisoning and food borne Bacillus cereus strains. Journal of Clinical Microbiology 40: 3053-3056.
Hoa, N. T., Baccigalupi, L., Huxham, A., Smertenko, A., Van, P. H., Ammendola, S., Ricca, E., and Cutting, S. M. (2000) Characterization of Bacillus species used for oral bacteriotherapy and bacterioprophylaxis of gastrointestinal disorders. Appl. Env. Microbiol. 66: 5241-5247.
Hoa, T. T., Duc, L. H., Isticato, R., Baccigalupi, L., Ricca, E., Van, P. H., and Cutting, S. M. (2001) Fate and dissemination of Bacillus subtilis spores in a murine model. Appl. Env. Microbiol. 67: 3819-3823.
Hong, H. A., Duc, L. H., and Cutting, S. M. (20055 The use of bacterial spore formers as probiotics. FEMS Microbiology Reviews 29: 813-835.
Hosoi, T., and Kiuchi, K. (2004) Production and probiotic effects of Natto. Ricca, E., Henriques, A. O. and Cutting, S. M. (eds). Wymondham, UK: Horizon Bioscience, pp. 143-154.
Jensen, G. B., Larsen, P., Jacobsen, B. L., Madsen, B., Wilcks, A., Smidt, L., and Andrup, L. (2002) Isolation and characterization of Bacillus cereus-like bacteria from faecal samples from greenhouse workers who are using Bacillus thuringiensis-based insecticides. Int Arch Occup Environ Health 75: 191-196.
Jensen, G. B., Hansen, B. M., Eilenberg, J., and Mahillon, J. (2003) The hidden lifestyles of Bacillus cereus and relatives. Environmental Microbiology 5: 631-640.
Keynan, A., and Evenchik, Z. (1969) Activation. In The bacterial spore. Gould, G. W. and Hurst, A. (eds). London: Academic Press, pp. 359-396.
Logan, N. A. (2004) Safety of aerobic endospore-forming bacteria. In Bacterial spore formers: probiotics and emerging applications. Ricca, B., Henriques, A. O. and Cutting, S. M. (eds). Norfolk, UK: Horizon Bioscience, pp. 93-106.
Mock, M., and Fouet, A. (2001) Anthrax. Annu. Rev. Microbiol. 55: 647-671.
Nagal, S., Okimura, K., Kaizawa, N., Ohki, K., and Kanatomo, S. (1996) Study on surfactin, a cyclic depsipeptide. II. Synthesis of surfactin B2 produced by Bacillus natto KMD 2311. Chem. Phar. Bull. (Tokyo) 44: 5-10.
Nakano, M. M., Dailly, Y. P., Zuber, P., and Clark, D. P. (1997) Characterization of anaerobic fermentative growth of Bacillus subtilis: identification of fermentation end products and genes required for growth. J Bacteriol 179: 6749-6755.
Nakano, M. M., and Zuber, P. (1998) Anaerobic growth of a “strict aerobe” (Bacillus subtilis). Annual Review of Microbiology 52: 165-190.
Nicholson, W. L., and Setlow, P. (1990) Sporulation, germination and outgrowth. In Molecular Biological Methods for Bacillus. Harwood., C. R. and Cutting., S. M. (eds). Chichester, UK.: John Wiley & Sons Ltd., pp. 391-450.
Nicholson, W. L. (2002) Roles of Bacillus endospores in the environment. Cellular and Molecular Life Sciences 59: 410-416.
Osipova, I. G., Sorokulova, I. B., Tereshkina, N. V., and Grigor'eva, L. V. (1998) Safety of bacteria of the genus Bacillus, forming the base of some probiotics. Zh Mikrobiol Epidemiol Immunobiol: 68-70.
Phelps, R. J., and McKillip, J. L. (2002) Enterotoxin production in natural isolates of Bacillaceae outside the Bacillus cereus group. Applied and Environmental Microbiology 68: 3147-3151.
Rowan, N. J., Deans, K., Anderson, J. G., Gemmell, C. G., Hunter, I. S., and Chaithong, T. (2001) Putative Virulence Factor Expression by Clinical and Food Isolates of Bacillus spp. after Growth in Reconstituted Infant Milk Formulae. App. Environ. Microbiol. 67: 3873-3881.
Salminen, S., von Wright, A., Morelli, L., Marteau, P., Brassart, D., de Vos, W. M., Fonden, R., Saxelin, M., Collins, K., Mogensen, G., Birkeland, S.-E., and Mattila-Sandholm, T. (1998) Demonstration of safety of probiotics—a review. International Journal of Food Microbiology 44: 93-106.
Sanders, M. E. (2003) Probiotics: considerations for human health. Nutr Rev 61: 91-99.
Sanders, M. E., Morelli, L., and Tompkins, T. A. (2003) Sporeformers as human probiotics: Bacillus, Sporolactobacillus, and Brevibacillus. Comprehensive Reviews in Food Science and Food Safety 2: 101-110.
SCAN (2000) Opinion of the Scientific Committee on Animal Nutrition on the safety of the use of Bacillus species in animal nutrition. European Commission, Health and Consumer Protection Directorate-General. (SCAN) Scientific Committee on Animal Nutrition.
SCAN (2001) http://ec.europa.eu/food/fs/sc/scan/out68_en.pdf. European Commission, Health and Consumer Protection Directorate-General. (SCAN) Scientific Committee on Animal Nutrition.
SCAN (2002) Opinion of the Scientific Committee on Animal Nutrition (SCAN) on the use of Bacillus licheniformis NCTC 13123 in feeding stuffs for pigs (product AlCare™). European Commission, Health and Consumer Protection Directorate-General. (SCAN) Scientific Committee on Animal Nutrition.
SCAN (2003a) On a generic approach to the safety assessment of microorganisms used in feed/food and feed/food production. A working paper open for comment. European Commission, Health and Consumer Protection Directorate-General. (SCAN) Scientific Committee on Animal Nutrition.
SCAN (2003b) Opinion of the Scientific Committee on Animal Nutrition, on the criteria for assessing the safety of microorganisms resistant to antibiotics of human clinical and veterinary importance. European Commission, Health and Consumer Protection Directorate-General. (SCAN) Scientific Committee on Animal Nutrition.
Standards, N.C.f.C.L. (1997) Performance standards for antimicrobial disk and dilution susceptibility tests for bacteria isolated from animals; approved standard-Second Edition. Approved standard M2-A5. Wayne, Pa.: NCCLS.
Tam, N. K., Uyen, N. Q., Hong, H. A., Duc le, H., Hoa, T. T., Serra, C. R., Henriques, A. O., and Cutting, S. M. (2006) The Intestinal Life Cycle of Bacillus subtilis and Close Relatives. J Bacteriol 188: 2692-2700.
Youngman, P., Perkins, J., and Losick, R. (1984) Construction of a cloning site near one end of Tn917 into which foreign DNA may be inserted without affecting transposition in Bacillus subtilis or expression of the transposon-borne erm gene. Plasmid 12: 1-9.

1-30. (canceled)

31. A method comprising:
(a) preparing a colourant, dye, food, food additive, food supplement or a cosmetic comprising preparing a composition comprising a Bacillus, or an extract from a Bacillus;
(b) colouring or dyeing by including or exposing to a Bacillus, or an extract from a Bacillus;
(c) administering to a human or animal a food, food supplement or a cosmetic comprising a Bacillus, or an extract from a Bacillus,
wherein the Bacillus has, or the extract is obtained from a Bacillus with, at least 95% 16S rRNA sequence identity to any of SEQ ID Nos 1 to 6.

32. A method according to claim 31, wherein the Bacillus is one selected from Bacillus spp. HU19 (NCIMB 41359), HU28 (NCIMB 41360), HU33 (NCIMB 41342) and HU36 (NCIMB 41361), or a derivative, variant or mutant of any thereof, where the 16S rRNA gene of the derivative, variant or mutant has at least 95% 16S rRNA sequence identity to that of any one of HU 19, HU 28, HU 33, and HU 36.

33. A method according to claim 31 wherein the food or food supplement is a food or food supplement for, or is administered to:
(i) a human;
(ii) a non-human farmed land animal; or
(iii) an aquatic animal.

34. A method according to claim 33 wherein the aquatic animal is a fish or a shell-fish.

35. A method according to claim 31, wherein consumption of the food or supplement results in adding colour to one or more tissues of the animal.

36. A method according to claim 31, wherein the spores and vegetative cells of the Bacillus are a different colour because of differential presence of at least one carotenoid in the spore and vegetative cells forms.

37. The method of claim 31, wherein:
(i) at least one carotenoid is present in the spore form, which is not present in the vegetative cell form; and/or
(ii) at least one carotenoid is present in the vegetative cell form, which is not present in the spore form.

38. The method of claim 31, wherein the carotenoid is a keto/hydroxyl-carotene derivative, astaxanthin, 4-ketozeaxanthin, echinenone, a hydroxyl-echinenone derivative, phenicoaxanthin, canthaxanthin, zeaxanthin, and/or α-carotene.

39. The method of claim 31 wherein the Bacillus has been genetically manipulated to express a gene and/or to inactivate a gene.

40. The method of claim 31 in which the Bacillus heterologously expresses one or more of the following existing carotenoid gene products:
a bicyclic beta cyclase which is a product of the crtY and/or crtL-b genes;
an epilson cyclases which is a product of the crtL-e gene;
a dehydrosqualene synthase which is a product of the crtM gene;
a dehydrosqualene desaturase which is a product of the crtN gene;
a phytoene desaturase which is a product of the crtI gene;
a phytoene desaturase which is a product of the Pds gene;
a ζ-carotene desaturase which is a product of the crtQ gene;
a ζ-carotene desaturase which is a product of the Zds gene;
a lycopene elongase which is a product of the crtEb; C50 carotenoid biosynthesis;
a zeaxanthin glucosylase which is a product of the crtX gene;
a C3 carotene hydroxylase (which is a product of crtZ like gene);
a C2 carotene hydroxylase (which is a product of the BcrtG gene);
a C4 oxygenase (which is a products of crtW like genes);
a C4 oxygenase (which are products of bkt like genes);
a zeaxanthin epoxidase which is a product of the zep1 gene;
a violaxanthin deepoxidase which is a product of the vde gene;
a capsanthin/capsorubin synthase which is a product of the ccs gene,
a β-carotene desaturase which is a product of the crtU gene;
a decaprenoxanthin synthase which is a product of the crtYe/Yf gene;
a carotenoid cleavage dioxygenase (CCD's) to yield aroma, flavour and/or a pharmaceutical related compound.

41. The method of claim 39, wherein the Bacillus has been genetically modified to introduce one or more of the genes of the mevalonate (MVA) pathway.

42. The method of claim 31, wherein the Bacillus has one or more of the following genes:
a ggpp synthase (crtE like) gene;
a phytoene synthase (crtB like) gene;
a three or four step phytoene desaturase (crtI like) gene;
a cyclase (probably mono-cyclase) (crtY like) gene;
a β-ring 3,3′ hydroxylase (crtZ like) gene; and/or
a β-ring 4,4′ oxygenase (ketolase), (crtW and crtO/bkt like) gene.

43. A method of producing a carotenoid, a metabolic precursor thereof or a derivative thereof comprising:
(i) growing vegetatively and/or producing spores of a Bacillus as defined in claim 31; and
(ii) extracting the carotenoid, metabolic precursor thereof, or the derivative thereof from the Bacillus.

44. A method according to claim 43, wherein the carotenoid is gastric resistant so that after incubation for one hour with simulated gastric fluid at least 50% of the carotenoid remains.

45. A Bacillus selected from a Bacillus with at least 95% 16S rRNA sequence identity to the sequence of any one of SEQ ID Nos 1 to 6, where the sores and vegetative cells of the Bacillus are a different colour because of differential presence of at least one carotenoid in the spore and vegetative cells forms

46. A Bacillus according to claim 45 which is a Bacillus selected from Bacillus spp. HU19 (NCIMB 41359), HU28 (NCIMB 41360), HU33 (NCIMB 41342) and HU36 (NCIMB 41361), or a derivative, variant or mutant of any thereof, where the derivative, variant or mutant has at least 95% 16S rRNA sequence identity to that of any one of HU 19, HU 28, HU 33, and HU 36.

47. A composition of matter selected from the group consisting of:
(a) a vaccine comprising a Bacillus as defined in claim 31 and which expresses a heterologous antigen;
(b) food-stuff, food additive, dye, colourant, cosmetic, nutraceutical or probiotic composition comprising a Bacillus, or an extract from a Bacillus, where the Bacillus is as defined in claim 31; and
(c) a pharmaceutical comprising a Bacillus as defined in claim 31, or an extract from such a Bacillus, and a pharmaceutically acceptable carrier or excipient.

48. A method of treating a human or animal body the method comprising administering a Bacillus as defined in 31, or a carotenoid extract from such a Bacillus, to the human or animal.

49. A method of detecting a stimulus comprising:
(i) providing spores of a Bacillus which are triggered when the stimulus is present to germinate to give vegetative cells, where the Bacillus is as defined in claim 36;
(ii) exposing the spores to test conditions under which it is desired to determine whether the stimulus is present or absent; and
(iii) detecting the presence or absence of the colour change resulting from germination of spores in order to determine the presence or absence of the stimulus.

50. A method of detecting an agent capable of modulating Bacillus growth, germination or sporulation, the method comprising:
(i) contacting a test agent with a Bacillus as defined in claim 36, the Bacillus; and
(ii) monitoring for a colour change or change in colour intensity.

51. A biosensor comprising a Bacillus and a support, where the Bacillus is as defined in claim 36

52. A method comprising:
(a) preparing a colourant, dye, food, food additive, food supplement or a cosmetic comprising preparing a composition comprising a Bacillus, or an extract from a Bacillus;
(b) colouring or dyeing by including or exposing to a Bacillus, or an extract from a Bacillus;
(c) administering to a human or animal a food, food supplement or a cosmetic comprising a Bacillus, or an extract from a Bacillus,
wherein the Bacillus, or the extract is obtained from a Bacillus, wherein the Bacillus is one whose spores and vegetative cells comprise different amounts of at least one carotenoid.
